SEQUENCE LISTING (8 sequences)

SEQ ID NO: 1 - DNA, 747 bp, Homo sapiens
atggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagatggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctga

SEQ ID NO: 2 - DNA, 1336 bp, Homo sapiens
gagagacacagagtccggcattggtcccaggcagcagttagcccgccgcccgcctgtgtgtccccagagccatggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctgagtgttgcccgccaccgccccgccctgccccctccagtcccccaccctgccgagaggactagtatggggtgggaggccccacccttctcccctaggcgctgttcttgctccaaagggctccgtggagagggactggcagagctgaggccacctggggctggggatcccactcttcttgcagctgttgagcgcacctaaccactggtcatgcccccacccctgctctccgcacccgcttcctcccgaccccaggaccaggctacttctcccctcctcttgcctccctcctgcccctgctgcctctgatcgtaggaattgaggagtgtcccgccttgtggctgagaactggacagtggcaggggctggagatgggtgtgtgtgtgtgtgtgtgtgtgtgtgtgtgtgtgcgcgcgcgccagtgcaagaccgagattgagggaaagcatgtctgctgggtgtgaccatgtttcctctcaataaagttcccctgtgacactcaaaaaaaaaaaaaaaaaaaaa

SEQ ID NO: 3 - protein, 248 aa, Homo sapiens [sequence omitted]

SEQ ID NO: 4 - protein, 248 aa, Homo sapiens [sequence omitted]

SEQ ID NO: 5 - protein, 46 aa, Homo sapiens
  BINDING (1)..(46): This peptide corresponds to residues 203-241 of 14-3-3sigma are involved in binding actin
ADLHTLSEDSYKDSTLIMQLLRDNLTLWTADNAGEEGGEAPQEPQS

SEQ ID NO: 6 - DNA, 19 bp, Homo sapiens
  misc_binding (1)..(19): This sequence corresponds to the target region of the 14-3-3sigma siRNA oligonucleotides
gtgaccatgtttcctctca

SEQ ID NO: 7 - DNA, 19 bp, Artificial Sequence
  Synthetic 14-3-3sigma siRNA oligonucleotide sequence
gtgaccatgtttcctctca

SEQ ID NO: 8 - DNA, 19 bp, Artificial Sequence
  Synthetic14-3-3sigma siRNA oligonucleotide sequence
tgagaggaaacatggtcac

(19) United States
(12) Patent Application Publication
(10) Pub. No.: US 2012/0329663 A1
(43) Pub. Date: Dec. 27, 2012

(54) 14-3-3 sigma as a Biomarker of Basal Cancer

(75) Inventors: Aaron T. Boudreau, Berkeley, CA (US); Mina J. Bissell, Berkeley, CA (US)

(73) Assignee: THE REGENTS OF THE UNIVERSITY OF CALIFORNIA, Oakland, CA (US)

(21) Appl. No.: 13/330,467

(22) Filed: Dec. 19, 2011

Related U.S. Application Data
(63) Continuation of application No. PCT/US2010/039276, filed on Jun. 18, 2010.
(60) Provisional application No. 61/218,872, filed on Jun. 19, 2009.

(51) Int. Cl. C12Q 1/68; C07H 21/02; C40B 30/04; C07H 21/00; G01N 33/53; C12N 15/63

(52) U.S. Cl. 506/9; 435/6.11; 435/6.12; 435/6.14; 435/7.1; 435/320.1; 536/24.5

(57) ABSTRACT

Methods and compositions for using 14-3-3sigma gene and protein as a highly sensitive and specific basal breast cancer biomarker, which when present, correlates with metastasis and poor outcome in independent patient cohorts. Methods and compositions for targeting 14-3-3sigma-regulated actin cytoskeletal interactions, activity and function may benefit patients having the basal-like breast cancer subtype.

### CROSS-REFERENCE TO RELATED APPLICATIONS

This application is a continuation of International Patent Application No. PCT/US2010/039276, filed on Jun. 18, 2010, which claims the benefit of priority to U.S. Provisional Patent Application No. 61/218,872, filed on Jun. 19, 2009, both of which are hereby incorporated by reference in their entirety.

### STATEMENT OF GOVERNMENTAL SUPPORT

This work was supported by Grant No. W81XWH-05-1-0339 awarded by Department of Defense Breast Cancer Research Program; and by Contract No. DE-AC02-05CH11231 awarded by the Department of Energy. The government has certain rights in this invention.

### REFERENCE TO SEQUENCE LISTING

This application incorporates by reference in its entirety the sequence listing attached in computer-readable and paper form.

### BACKGROUND OF THE INVENTION

1. Field of the Invention

This invention is in the field of breast cancer biomarkers for basal cancers. More specifically, the present invention relates to the detection, validation and inhibition of a basal cancer marker.

2. Related Art

Molecular profiling, immunohistochemical characterization, and clinical evaluation irrefutably demonstrate that breast cancer is not a single disease, but rather loosely describes a pathological state with exceedingly complex phenotypic and behavioral heterogeneity. While clinicians routinely categorize primary breast tumors based on differentiation state, mitotic index, and the expression of biomarkers such as HER2/ErbB2, estrogen and progesterone receptors (ER and PgR, respectively) to determine treatment options for patients, gene expression profiling experiments have consistently shown that tumors can be generally classified into at least five groups which recapitulate many clinical features of breast cancer heterogeneity and which have associated prognostic value with respect to patient outcome and chemotherapeutic response. Among these groups, basal-like breast cancer (BBC) accounts for roughly 15% of invasive breast carcinomas and although it lacks a formalized definition, BBC is characterized by high tumor grade, the expression of several markers normally restricted to the basal/myoepithelial compartment, epidermal growth factor receptor (EGFR) amplification in approximately 50% of cases, and the absence of ER and HER2 which are associated with luminal and HER2-amplified tumors, respectively. As BBC tumors are not responsive to agents targeting ER and HER2 and, unlike luminal and HER2-amplified tumors, there are no consensus “functional biomarkers” contributing to BBC disease progression, this subtype of breast cancer presents the most challenging case for current clinical treatment and detection, and major effort has been placed on understanding the molecular basis governing BBC progression.

Early studies in HCT116 colorectal cancer cells identified 14-3-3sigma as a p53 transcriptional target which regulates the G2/M checkpoint by sequestering cyclin B1 and cdc2 to the cytosol, and further showed that 14-3-3sigma is required to prevent mitotic catastrophe following DNA damage, implicating 14-3-3sigma as a tumor suppressor and that 14-3-3sigma functions is down-regulated in breast cancer (Mhawech P: 14-3-3 proteins—an update. 2005 Cell Res, 15(4):228-236; Vercoutter-Edouart A S, Lemoine J, Le Bourhis X, Louis H, Boilly B, Nurcombe V, Revillion F, Peyrat J P, Hondermarck H: Proteomic analysis reveals that 14-3-3sigma is down-regulated in human breast cancer cells. 2001 Cancer Res, 61(1):76-80).

14-3-3sigma expression is elevated in pancreatic, colorectal, head and neck, and endometrial carcinomas, as well as in various epithelial cancers with squamous differentiation. Intriguingly, overexpression of 14-3-3sigma in the aforementioned HCT116 colorectal cancer cells promotes cell motility in migration assays which, in conjunction with an increased presence of 14-3-3sigma at the invasive front of tumors in patients, suggests an additional role for 14-3-3sigma independent of cell cycle regulation in facilitating colorectal tumor progression to invasive carcinoma (Ide M, Saito K, Tsutsumi S, Tsuboi K, Yamaguchi S, Asao T, Kuwano H, Nakajima T: Over-expression of 14-3-3sigma in budding colorectal cancer cells modulates cell migration in the presence of tenascin-C. 2007 Oncol Rep, 18(6):1451-1456). Forced expression of 14-3-3sigma in PANC-1 pancreatic cancer cell lines also promotes cell migration and invasion independently of altering proliferation (Neupane D, Korc M: 14-3-3sigma Modulates pancreatic cancer cell survival and invasiveness. 2008 Clin Cancer Res, 14(23):7614-7623).

Whether 14-3-3sigma silencing in breast tumors is as prevalent as initially reported has been the subject of recent controversy (Moreira J M, Ohlsson G, Rank F E, Celis J E: Down-regulation of the tumor suppressor protein 14-3-3sigma is a sporadic event in cancer of the breast. 2005 Mol Cell Proteomics, 4(4):555-569), as in early papers describing declined 14-3-3sigma in breast cancer, expression was normalized to non-malignant cell lines derived from reduction mammoplasty (primary HMECs, MCF-10A, and 184) (Vercoutter-Edouart A S, Lemoine J, Le Bourhis X, Louis H, Boilly B, Nurcombe V, Revillion F, Peyrat J P, Hondermarck H: Proteomic analysis reveals that 14-3-3sigma is down-regulated in human breast cancer cells. 2001 Cancer Res, 61(1):76-80). Primary mammary epithelial cells rapidly adopt myoepithelial/basal-like gene expression profiles when cultured on tissue culture plastic in the absence of selective growth media. This is an important consideration and a caveat to the design of previous experiments, as 14-3-3sigma is highly expressed in breast myoepithelial cells and is only marginally expressed in luminal epithelial cells—a trend consistent in pathologically normal tissue, in premalignant lesions, and in normal tissue adjacent to primary breast tumors (Moreira et al, 2005 Mol Cell Proteomics, 4(4):555-569; Simpson P T, Gale T, Reis-Filho J S, Jones C, Parry S, Steele D, Cossu A, Budroni M, Palmieri G, Lakhani S R: Distribution and significance of 14-3-3sigma, a novel myoepithelial marker, in normal, benign, and malignant breast tissue. 2004 J Pathol, 202(3):274-285). Correspondingly, primary cultures derived from the tissue of a breast cancer patient with weak 14-3-3sigma tumor immunoreactivity have robust 14-3-3sigma expression after 6 passages in culture, indicating rapid population drift towards myoepithelial gene expression profile (Moreira et al, 2005 Mol Cell Proteomics, 4(4):555-569). Given that tumors stain relatively homogenous for 14-3-3sigma (Moreira et al, 2005 Mol Cell Proteomics, 4(4):555-569) and that >80% of tumors lack basal/myoepithelial-like differentiation (Lakhani S R, O'Hare M J: The mammary myoepithelial cell—Cinderella or ugly sister?, 2001 Breast Cancer Res, 3(1):1-4), comparing gene expression profiles between a homogenous “non-myoepithelial” population and a homogenous “myoepithelial” population would artificially identify several lineage-specific markers including 14-3-3sigma, and the previously reported 14-3-3sigma downregulation frequency of >80% is in good agreement with the frequency of tumors lacking basal differentiation.

Previous studies describing 14-3-3-mediated actin reorganization have highlighted a number of actin cytoskeletal signaling pathways under regulation by various 14-3-3 family members with a common theme of 14-3-3-dependent cofilin regulation during cell migration. The 14-3-3 family of molecular scaffolds is comprised of seven known mammalian isoforms (β, γ, ε, η, σ, θ/τ, and ζ), and regulate pathways involved in growth factor signaling and cell cycle progression through binding and sequestering the subcellular distribution of numerous ligands (Aitken A: 14-3-3 proteins: a historic overview. 2006 Semin Cancer Biol, 16(3):162-172; Porter G W, Khuri F R, Fu H: Dynamic 14-3-3/client protein interactions integrate survival and apoptotic pathways. 2006 Semin Cancer Biol, 16(3):193-202). 14-3-3sigma is widely described as a breast tumor suppressor due to the high frequency of 14-3-3sigma downmodulation in breast cancer (Urano T, Saito T, Tsukui T, Fujita M, Hosoi T, Muramatsu M, Ouchi Y, Inoue S: Efp targets 14-3-3 sigma for proteolysis and promotes breast tumour growth. 2002 Nature, 417(6891):871-875) and its function as an enforcer of the G2/M checkpoint [23, 24]. However, 14-3-3sigma silencing was demonstrated to not occur as frequently in breast cancer as initially proposed (Moreira J M, Ohlsson G, Rank F E, Celis J E: Down-regulation of the tumor suppressor protein 14-3-3sigma is a sporadic event in cancer of the breast. 2005 Mol Cell Proteomics, 4(4):555-569), and rather was found in a pilot study to be highly expressed in 10 of 12 cases of BBC (Simpson P T, Gale T, Reis-Filho J S, Jones C, Parry S, Steele D, Cossu A, Budroni M, Palmieri G, Lakhani S R: Distribution and significance of 14-3-3sigma, a novel myoepithelial marker, in normal, benign, and malignant breast tissue. 2004 J Pathol, 202(3):274-285), in agreement with 14-3-3sigma serving as a biomarker of myoepithelial cells in the normal breast. These studies provide evidence that 14-3-3sigma may have as yet undescribed functions related to breast cancer progression, particularly in the BBC subtype, and that our cell culture model resembling BBC progression may provide clues as to what these functions entail.

In addition to the cofilin pathway, a recent study found several 14-3-3 isoforms bind to the AKT substrate Kank, and that Kank inhibits the insulin- and AKT-dependent activation of RhoA and subsequent actin cytoskeletal dynamics through interaction with 14-3-3 (Kakinuma N, Roy B C, Zhu Y, Wang Y, Kiyama R: Kank regulates RhoA-dependent formation of actin stress fibers and cell migration via 14-3-3 in PI3K-Akt signaling. 2008 J Cell Biol, 181(3):537-549). Activated AKT can interact with both actin and with 14-3-3sigma [66], and 14-3-3sigma has been shown to negatively regulate AKT in MCF-7 cells and to inversely correlate with AKT activity in tumors (Yang H, Wen Y Y, Zhao R, Lin Y L, Fournier K, Yang H Y, Qiu Y, Diaz J, Laronga C, Lee M H: DNA damage-induced protein 14-3-3 sigma inhibits protein kinase B/Akt activation and suppresses Akt-activated cancer. 2006 Cancer Res, 66(6):3096-3105). These studies suggest the existence of an alternative 14-3-3-dependent actin regulatory pathway by which AKT activated during chemotaxis is recruited to actin and may locally activate RhoA, but excessive AKT activity is suppressed directly by 14-3-3sigma or through AKT-dependent phosphorylation and activation of Kank, which downmodulates RhoA activity via 14-3-3 interaction.

14-3-3sigma has been previously implicated by Simpson P T, Gale T, Reis-Filho J S, Jones C, Parry S, Steele D, Cossu A, Budroni M, Palmieri G, Lakhani SR: Distribution and significance of 14-3-3sigma, a novel myoepithelial marker, in normal, benign, and malignant breast tissue. 2004 J Pathol, 202(3):274-285 as an adjunct biomarker of basal-like breast cancer, being highly expressed in 10 of 12 tumors showing basal-like differentiation. However, 14-3-3sigma interaction and regulation of actin polymerization was previously unknown.

### BRIEF SUMMARY OF THE INVENTION

14-3-3sigma was initially described as a breast tumor suppressor silenced in about 80% of tumors. However, in two independent culture models which recapitulate features of basal-like breast cancer progression, we show 14-3-3sigma expression correlates with acquisition of tumorigenicity and invasiveness, and that 14-3-3sigma coordinates directional cell migration by directly inhibiting actin polymerization (in vitro and in situ) downstream of canonical signaling pathways, thus defining regions of actin assembly within cells. By immunohistochemical analysis, 14-3-3sigma emerged as a highly sensitive and specific basal breast cancer biomarker, which when present, correlates with metastasis and poor outcome in independent patient cohorts. These data suggest that targeting 14-3-3sigma-regulated actin cytoskeletal dynamics may benefit patients having the basal-like breast cancer subtype.

Thus the present invention provides an assay for diagnosis of basal-like breast cancer comprising the steps of (a) obtaining a breast tissue sample, and (b) detecting a positive and/or elevated level of 14-3-3sigma expression in said breast tissue sample as compared to a reference, whereby such positive and/or elevated detection is a diagnosis of basal-like breast cancer.

In one embodiment, the detecting can be carried out by fluorescence in situ hybridization (FISH), polymerase chain reaction (PCR), or RT-PCR, to detect 14-3-3sigma expression levels. In another embodiment, the detecting is by an immunohistochemical (IHC) analysis to detect 14-3-3sigma protein levels. A positive level of 14-3-3sigma is detected in IHC analysis if the 14-3-3sigma level detected in said breast tissue sample is greater than or equal to the levels observed in a myoepithelial cell tissue sample of the normal breast.

In one embodiment, the present invention further provides a kit containing reagents to carry out the assay to detect basal-like cancer including instructions for carrying out any necessary steps.

In another embodiment, the present invention describes a method for identifying an ER-negative cancer patient with poor prognosis, comprising: (a) measuring the expression level of 14-3-3sigma (SFN) gene in a sample from the patient; and (b) comparing the expression level of said gene from the patient with the expression level of the gene in a normal tissue sample or a reference expression level, whereby an increased expression level in said gene indicates a patient with poor prognosis.

In another embodiment, a compound to treat patients with elevated 14-3-3sigma expression, wherein the compound is a 14-3-3sigma inhibitor. The 14-3-3sigma inhibitor compound can be a small molecule that interferes with 14-3-3sigma function, a viral vector producing a nucleic acid sequence that inhibits 14-3-3sigma, an aptamer, an antisense oligonucleotide, a siRNA oligonucleotide, or a peptide. In one embodiment, the siRNA oligonucleotide has a sequence of SEQ ID NOS: 7 and 8.

In another embodiment, a method for causing actin bundling and inhibition of actin depolymerization in a cell comprising the steps of (a) providing a 14-3-3sigma inhibitor, and (b) delivering to the cell an effective amount of the 14-3-3sigma inhibitor, whereby the inhibitor blocks 14-3-3sigma interaction with actin and thereby causes acting bundling or inhibits actin depolymerization. The 14-3-3sigma inhibitor can be a blocking or dominant-negative peptide, protein, or peptidomimetic; an antisense oligonucleotide; a siRNA oligonucleotide; a small molecule that interferes with 14-3-3sigma function; a viral vector producing a nucleic acid sequence that inhibits 14-3-3sigma; or an aptamer.

The present invention further provides a method for inhibiting metastasis of a cell in a patient comprising administering a therapeutic, wherein the therapeutic interferes with 14-3-3-mediated interaction with actin, 14-3-3 dimerization, or wherein the therapeutic stabilizes interaction between 14-3-3sigma and proteins other than actin.

And in another embodiment, a method of treatment to reduce or inhibit invasiveness of malignant cells and reduce tumor volume and/or formation comprising the steps of (1) providing a 14-3-3sigma inhibitor capable of blocking 14-3-3sigma interaction with actin and (b) administering an effective amount of the 14-3-3sigma inhibitor to block 14-3-3sigma interaction with actin, whereby blocking the interaction results in a reduction or inhibition of invasiveness of malignant cells and a reduction in tumor volume and/or formation. The 14-3-3sigma inhibitor can be a blocking or dominant-negative peptide, protein, or peptidomimetic; an antisense oligonucleotide; a siRNA oligonucleotide; a small molecule that interferes with 14-3-3sigma function; a viral vector producing a nucleic acid sequence that inhibits 14-3-3sigma; or an aptamer.

### BRIEF DESCRIPTION OF THE DRAWINGS

FIG. 1: 14-3-3sigma increases during HMT-3522 basal-like breast tumor progression, is highly expressed at the tumor invasive front in vivo, and contributes to motility and invasiveness. (A) Western blot analysis of lysates from the HMT-3522 series when grown on two-dimensional tissue culture (2D) or on top of three-dimensional lrECM (3D lrECM), showing 14-3-3sigma gradually increases in the series when cells are grown both in 2D and in 3D lrECM. Actin and E-cadherin are provided as loading controls. (B) 14-3-3sigma immunohistochemical staining in a T4-2 xenograft, showing strong 14-3-3sigma immunoreactivity throughout the tumor with intensification occurring at the tumor invasive front. The surrounding mouse stroma is negative for 14-3-3sigma staining (left). A heat map representation of the same tumor section emphasizes the 14-3-3sigma distribution (right). Scale bar: 100 μm. (C) T4-2 cells expressing shRNA hairpins targeting 14-3-3sigma (sh-14-3-3sigma) have greater than 80% reduction on the protein level (left) relative to cells expressing scrambled shRNAs (sh-scr), have decreased motility in scratch assays (middle), and have decreased invasiveness through lrECM-coated transwell inserts (right). Error bars represent the standard error of the mean (SEM), statistical significance was calculated using a two-tailed Student's t test (* p<0.05, ** p<0.01).

FIG. 2: 14-3-3sigma inhibits actin polymerization in a ligand-binding mechanism. (A) T4-2 cells with reduced 14-3-3sigma (sh-14-3-3sigma) have more polymerized actin than cells expressing scrambled hairpins (sh-scr) as visualized by phalloidin staining. There is no difference in actin protein levels, and F-actin inhibition by 14-3-3sigma occurs downstream of canonical cytoskeletal signaling (see FIG. 7). Scale bar: 20 μm. (B) Schematic showing helices involved in wild-type 14-3-3sigma (14-3-3σ^{WT}) dimerization and ligand interaction, as well as regions influenced by a C-terminal truncation (14-3-3σ^{ΔC}). The mutant 14-3-3sigma is able to dimerize, but is missing two helices known to interact with ligand (not shown to scale). (C) Phalloidin stains, showing the increased actin polymerization in sh-14-3-3sigma cells can be rescued with forced expression of 14-3-3σ^{WT}, but not an irrelevant gene (Gus) nor the mutant 14-3-3σ^{ΔC}. Scale bar: 20 μm. (D) Quantification of the F-actin intensity per cell, relative to the intensity of sh-scr cells. Error bars represent the standard error of the mean (SEM), statistical significance was calculated using a one-way analysis of variance (ANOVA) with Bonferroni's multiple comparison post-test (* p<0.05, ** p<0.01).

FIG. 3: 14-3-3sigma interacts directly with actin and inhibits its polymerization into F-actin in vitro and in situ to define the boundary of actin architecture within cells. (A) A GST pull-down showing that recombinant GST-14-3-3sigma, but not GST tag, interacts directly with purified actin in vitro. (B) G-actin pretreatment with GST-14-3-3sigma inhibits subsequent polymerization into F-actin. This was observed by (i) fractionating the residual G-actin from F-actin following polymerization, and by (ii) polymerizing AlexaFluor488-conjugated G-actin (488-G-actin) and imaging F-actin fibrils by confocal microscopy. Depicted is the maximum intensity projection of 20 superimposed fields. Quantification (iii) is the residual G-actin pool remaining following polymerization, relative to pretreatment with GST. Error bars represent the standard error of the mean (SEM), statistical significance was calculated using a two-tailed Student's t test (*** p<0.001). (C) In situ actin polymerization was measured by permeabilizing cells with saponin, introducing 488-G-actin in the presence of ATP, then counterstaining endogenous F-actin with phalloidin. Pretreatment with GST-14-3-3sigma significantly inhibits 488-G-actin incorporation in cells with low endogenous 14-3-3sigma (BT549). Quantification is the ratio of incorporated 488-G-actin to endogenous F-actin, relative to values obtained by pretreating 488-G-actin with GST in the respective cell lines. Error bars represent the standard error of the mean (SEM), statistical significance was calculated using a two-tailed Student's t test (*** p<0.001, ns p>0.05). (D) F-actin and 14-3-3sigma subcellular distributions are mutually-exclusive, as shown by XY, YZ, and XZ projections. Scale bar: 5 μm.

FIG. 4: 14-3-3sigma polarizes away from the cell leading edge during directional migration to allow dynamic actin polymerization and reorganization. (A) F-actin (LifeAct-mCherry) and 14-3-3sigma (YFP-14-3-3sigma) dynamics during cell migration. Most 14-3-3sigma localizes away from the leading edge of the cell and defines the boundary of actin architecture, while rapid F-actin remodeling occurs either as lamellipod extension and retraction at the leading edge or as actin turnover at the trailing edge of the cell. (B) Differential heat map showing 14-3-3sigma subcellular distribution relative to F-actin in the frames from (A), emphasizing that 14-3-3sigma polarizes away from the leading edge of the cell during directional migration. Scale Bar: 10 μm

FIG. 5: 14-3-3sigma expression correlates with decreased patient survival, increased breast cancer recurrence, and with tumor dissemination. A) Kaplan-Meier survival analysis of the Netherlands Cancer Institute (NKI) and the USCF cohorts for patients grouped by 14-3-3sigma expression. (B) Kaplan-Meier recurrence-free survival analysis of the NKI and the USCF patient cohorts for patients grouped by 14-3-3sigma expression. (C) Kaplan-Meier metastasis-free survival analysis of the Netherlands NKI and the USCF patient cohorts for patients grouped by 14-3-3sigma expression. P values for all survival curves were calculated using the log-rank test

FIG. 6: 14-3-3sigma increases during MCF10 tumor progression and contributes to cell motility and stellate morphogenesis. (A) Western blot analysis of lysates from the MCF10 series. Actin and E-cadherin are provided as loading controls. 14-3-3sigma gradually increases in the series during tumor progression, as is the case for the HMT-3522 series. (B) MCF10DCIS.com cells expressing shRNA hairpins (sh-14-3-3sigma) targeting 14-3-3sigma have greater than 80% reduction on the protein level (left) relative to a scrambled shRNA (sh-scr), have decreased motility in scratch assays (middle), and have decreased numbers of invasive and migratory projections per colony when cultured on 3D lrECM (right). Error bars represent the standard error of the mean (SEM), statistical significance was calculated using a two-tailed Student's t test (** p<0.01, *** p<0.001).

FIG. 7: 14-3-3sigma perturbation in T4-2 cells does not influence actin cytoskeletal signaling nor induce epithelial to mesenchymal transition. A comparison between T4-2 cell populations expressing shRNAs targeting 14-3-3sigma or a scrambled control shows no differences in cytoskeletal signaling activity, suggesting that 14-3-3sigma inhibits actin polymerization downstream of these pathways. This also occurs in the absence of epithelial to mesenchymal transition.

FIG. 8: YFP-14-3-3sigma and LifeAct-mCherry distributions are mutually-exclusive and are comparable to their endogenous counterparts, while untagged mCherry is distributed throughout the cell. (A) YFP-14-3-3sigma is localized throughout the cytoplasm and is largely excluded from the nucleus (similar to endogenous 14-3-3sigma), while mCherry is localized throughout the cell. (B) In contrast to parental mCherry, LifeAct-mCherry is distributed almost entirely to F-actin rich regions (similar to phalloidin staining) and is mutually exclusive with respect to YFP-14-3-3sigma, validating the live cell probes behave similar to their endogenous counterparts and are suitable for spatiotemporal localization studies. Scale bar: 10 μm.

FIG. 9: 14-3-3sigma subcellular polarity persists throughout cell migration. Cells expressing YFP-14-3-3sigma and LifeAct-mCherry and which are actively migrating after 24 hours still have polarized 14-3-3sigma distribution away from the leading edge of the cell, suggesting that continual (rather than transient) 14-3-3sigma polarization occurs during cell migration. Scale bar: 20 μm.

FIG. 10: Endogenous 14-3-3sigma polarizes away from F-actin present at the leading edge of a cell during migration. T4-2 cells were grown to a confluent monolayer, scratched with a pipet tip, and migratory cells were fixed and stained 12 hours later to measure endogenous F-actin and 14-3-3sigma distributions. Migratory cells facing the scratch area are polarized such that 14-3-3sigma is away from the leading edge of cell migration, whereas stationary cells far from the scratch area show no 14-3-3sigma polarization. Scale bar: 20 μm.

FIG. 11: Patients with 14-3-3sigma-positive tumors have worse 5-year prognosis than patients with 14-3-3sigma-negative tumors. Recurrence-free and metastasis-free Kaplan-Meier survival curves for the Institute of Cancer Research cohort grouped by 14-3-3sigma immunoreactivity. 14-3-3sigma positivity (14-3-3sigma high) correlates with shorter 5-year recurrence-free and metastasis-free patient survival than 14-3-3sigma low patients. P values for survival curves were calculated using the log-rank test.

Table 1: Correlations between 14-3-3sigma immunoreactivity and clinic-histological parameters in 245 cases of invasive breast carcinoma.

### DETAILED DESCRIPTION OF THE PREFERRED EMBODIMENT

In the present study, we discovered a novel function for 14-3-3sigma in coordinating cell invasiveness and motility within both the HMT-3522 and the MCF10 progression series through directly regulating actin dynamics downstream of canonical cytoskeletal signaling pathways. 14-3-3sigma directly inhibits actin polymerization in vitro and in situ to define the boundaries of microfilament architecture within cells, and in migratory cells, 14-3-3sigma polarizes away from the leading edge to allow the dynamic actin remodeling necessary for directional migration.

The present invention provides compositions and methods that are based on the novel finding of a direct interaction between 14-3-3sigma and actin downstream or independent of these pathways, in that 14-3-3sigma alone can bind to actin and inhibits its polymerization. To our knowledge, this is the first biochemical demonstration of any 14-3-3 family member regulating actin dynamics independent of other actin-regulatory factors.

Thus, in one embodiment, 14-3-3sigma is contemplated to be a biomarker for basal, or basal-like cancers in any epithelial tissue showing elevated 14-3-3sigma expression including but not limited to tissues such as breast, ovary, prostate, lung, pancreatic, bladder, colorectal, endometrial, head and neck, skin, squamous, and other epithelial and myoepithelial tissues.

In one embodiment, 14-3-3sigma is a marker of basal or basal-like breast cancer. BBC tumors are aggressive in their pathology, correlate with poor patient outcome, and as they are negative for ER and HER2 and lack consensus biomarkers for their detection, BBC cases have limited options for current clinical management. A proposed BBC histological surrogate is the “triple-negative” (ER-, PR- and HER2-negative) immunoprofile, however it is well accepted that while triple-negative and BBC tumors do share some degree of pathological overlap, they are not interchangeable terms. Furthermore, hormone receptor and HER2 staining is not without its caveats, as heterogeneity within tumors and technical differences in tissue processing and interpretation can yield false negatives—presence of a specific marker is much preferred over absence of all markers. Other common BBC criteria include ER-negativity and positivity for basal cytokeratins (Ck5/6, Ck14, Ck17), EGFR, or other myoepithelial markers such as p63 and P-cadherin, though many of these markers lack in sensitivity, specificity, or it is unclear how they could be drivers of tumorigenesis rather than passengers.

In a tissue microarray (TMA) spotted with tumor biopsies from 245 cases of invasive breast carcinoma, we show 14-3-3sigma is a BBC histological biomarker with high sensitivity (70%; 16/23 basal tumors positive) and specificity (91%; 15/164 non-basal tumors positive). When used in conjunction with ER negativity, 14-3-3sigma positivity provided a predictive prognostic value of 94% (<6% false positives) for correctly identifying BBC cases. 14-3-3sigma expression was found to be highest in patients with basal-like breast cancer, having positive correlation with every basal histological marker investigated and inverse correlation with luminal biomarkers. This sensitivity is superior to Ck5/6 and EGFR, both of which are shown previously to detect only half of basal breast cancers, and neither ER nor EGFR are specific to basal tumors (Nielsen T O, Hsu F D, Jensen K, Cheang M, Karaca G, Hu Z, Hernandez-Boussard T, Livasy C, Cowan D, Dressler L et al: Immunohistochemical and clinical characterization of the basal-like subtype of invasive breast carcinoma. 2004 Clin Cancer Res, 10(16):5367-5374). It should be noted that while the Nielsen scoring criteria we used to classify basal tumors is 100% specific, it is only 75% sensitive, meaning we may have underestimated both the sensitivity and the specificity of 14-3-3sigma immunoreactivity as a BBC biomarker. Further validation in archival tissue molecularly classified as basal-like by cDNA microarray analysis could be warranted in order to more accurately assess 14-3-3sigma prognostic value.

Correspondingly, 14-3-3sigma immunoreactivity correlates with lower 5-year metastasis-free and recurrence-free survival within the TMA cohort, and 14-3-3sigma expression similarly correlates with tumor relapse, metastasis, and poor survival in two independent patient cohorts. We show that rather than being a breast tumor suppressor in the basal subtype, 14-3-3sigma represents the first “functional biomarker” of BBC which may contribute directly to tumor dissemination by modulating actin dynamics, making 14-3-3sigma a promising target of therapeutic intervention for patients with BBC.

In two independent isogenic cell culture models recapitulating many features of BBC progression, we identify 14-3-3sigma as a protein that becomes highly expressed as cells lines become more aggressive and tumorigenic, and show that 14-3-3sigma contributes to directional cell migration by directly interacting with actin and inhibits the polymerization of G-actin into F-actin to define actin assembly boundaries within cells. Furthermore, we validate 14-3-3sigma as a BBC biomarker in vivo, and show that 14-3-3sigma correlates with metastasis, relapse, and poor survival in multiple patient cohorts, suggesting that this protein may represent a BBC functional biomarker that contributes to malignant progression by modulating actin dynamics.

We show in three independent patient cohorts (one tissue microarray and two cDNA microarray datasets based on different microarray platforms, all from different institutes) that high 14-3-3sigma robustly correlates with poor patient outcome. While this is most likely due to 14-3-3sigma being a biomarker of a breast cancer subtype that is inherently aggressive in pathology, we believe 14-3-3sigma also contributes to BBC metastatic progression by modulating actin homeostasis, and we show that 14-3-3sigma follows tumorigenesis in independent BBC progression models. Based on these findings, we advocate 14-3-3sigma may represent a BBC functional biomarker that contributes to tumor pathology and progression by regulating cell motility, and ultimately tumor dissemination, via the actin cytoskeleton. Our data do not dispute the literature that 14-3-3sigma is frequently downregulated in breast cancer; rather, our data suggest that “silencing” of 14-3-3sigma in breast cancer is contingent on whether tumors show basal-like differentiation or luminal differentiation. In summary, this study presents 14-3-3sigma as an excellent functional biomarker of basal-like breast cancer, and suggests that personalized treatment targeting 14-3-3sigma-regulated actin cytoskeletal dynamics may be of benefit for patients with pre-metastatic disease.

Diagnostics Targeting 14-3-3sigma

Therefore, in some embodiments, 14-3-3sigma can be used as a predictive marker or serum marker for basal breast cancer detection. Assessment of amplification at 1q36.11 can be readily detected by methods known in the art. In another embodiment, a prognostic method for predicting the outcome of a patient by detection of 14-3-3sigma overexpression in a patient tissue or biopsy. Thus, detection of increased expression of 14-3-3sigma indicates the presence of aggressive or metastatic cancers, i.e., the presence of cells in the tissue that will increase tumor progression and metastasize to other tissues.

As shown in the Examples, 14-3-3sigma was determined to be involved in acquisition of invasiveness in culture in basal breast cancers. Proteomic profiling of the HMT-3522 progression series (Yan Y, Weaver V M, Blair I A: Analysis of protein expression during oxidative stress in breast epithelial cells using a stable isotope labeled proteome internal standard. 2005 J Proteome Res, 4(6):2007-2014; current study) independently identified 14-3-3sigma (SFN, HME1) as a protein expressed greater than two-fold higher in malignant T4-2 cells relative to their nonmalignant counterpart S1 cells. These results show that 14-3-3sigma has an active role in the acquisition of invasiveness of the premalignant cells, in addition to promoting the invasiveness of malignant cells.

In a preferred embodiment, 14-3-3sigma, which is also known as the gene stratifin (SFN), is a gene target for development of therapeutics and diagnostic assays. In one embodiment, an assay to detect elevated 14-3-3sigma expression as a predictor of invasive basal-like breast cancer. In such an assay, elevated 14-3-3sigma expression can be detected using methods known in the art. It is contemplated that elevated 14-3-3sigma expression can be detected in a subject by testing various tissues and bodily fluids, including but not limited to tissue biopsy, blood and serum.

In another embodiment, elevated 14-3-3sigma expression is detected using fluorescent in situ hybridization (FISH) to detect human chromosomal 1p36.11 amplification or 14-3-3sigma amplification. For example, one can create probes that hybridize to the 1p36.11 region or the genomic sequence flanking 14-3-3sigma in GenBank Accession No. NC_{—}000001.9 GI:89161185, hereby incorporated by reference. Probes can be created based upon the sequences of 14-3-3sigma using methods known in the art. In one embodiment, one can create probes for sequences described at GenBank Accession No: CR541926 (gi149456806), a Homo sapiens full open reading frame cDNA clone RZPDo834H1133D for gene SFN, stratifin (gi|49456806|emb|CR541926.1|[49456806]) (SEQ ID NO:1), hereby incorporated by reference. Other useful sequences for making probes and other sequences in the present invention include but are not limited to, NM_{—}006142.3 GI:45238846 (Homo sapiens stratifin SFN mRNA) (SEQ ID NO: 2); hereby incorporated by reference. In a specific embodiment, probes can be created by methods known in the art based upon the sequence of 14-3-3sigma in SEQ ID NOs: 1 or 2.

The expression level of a gene encoding 14-3-3sigma can be measured using an oligonucleotide derived from the nucleotide sequence of SEQ ID NO:1 or 2. In another embodiment, elevated 14-3-3sigma expression is detected using a PCR assay to detect 14-3-3sigma amplification. Primers can be created using the sequences of SEQ ID NOs: 1 or 2 or the GenBank cDNA or genomic sequences incorporated by reference, to detect 14-3-3sigma expression and amplification. Elevated expression of 14-3-3sigma can be detected by methods such as gel electrophoresis, comparative genomic hybridization or using tissue or cDNA microarrays. As is known in the art, primers or oligonucleotides are generally 15-40 bp in length, and usually flank unique sequence that can be amplified by methods such as polymerase chain reaction (PCR) or reverse transcriptase PCR. In yet another embodiment, elevated 14-3-3sigma expression is detected using an RT-PCR assay to detect 14-3-3sigma transcription levels.

The expression level of a gene is measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

The expression level of a gene is measured from a sample from the patient that comprises essentially a cancer cell or cancer tissue of a cancer tumor. Such methods for obtaining such samples are well known to those skilled in the art. When the cancer is breast cancer, the expression level of a gene is measured from a sample from the patient that comprises essentially a breast cancer cell or breast cancer tissue of a breast cancer tumor.

In some embodiments, the method further comprises measuring the expression level of 14-3-3sigma of the patients in order to determine whether the patient is an 14-3-3sigma-positive patient. Methods of assaying for 14-3-3sigma protein (SEQ ID NO:4) overexpression include methods that utilize immunohistochemistry (IHC) and methods that utilize fluorescence in situ hybridization (FISH). An example of a commercially available IHC test is PathVysion® (Vysis Inc., Downers Grove, Ill.). An example of a commercially available FISH test is DAKO HercepTest® (DAKO Corp., Carpinteria, Calif.).

In a preferred embodiment, elevated 14-3-3sigma expression is detected using an immunochemical assay to detect 14-3-3sigma protein levels. Anti-14-3-3sigma specific antibodies can be made by general methods known in the art. A preferred method of generating these antibodies is by first synthesizing peptide fragments. Peptide fragments can also be generated by using short sequences of the 14-3-3sigma protein, found at GenBank Accession Nos. CAG46724 GI:49456807; CAG46703 GI:49456765; AAH01550 GI:630737; NP_{—}006133 GI:5454052, all of which are hereby incorporated by reference. The 14-3-3sigma protein sequence can be found in GenBank Accession No: CAG46724.1 GI:49456807 (SEQ ID NO:4), which is hereby incorporated by reference and at Swiss-Prot P31947.1 GI:398953 (SEQ ID NO:3), the contents of the entry which are hereby incorporated by reference.

Since synthesized peptides are not always immunogenic by their own, the peptides should be conjugated to a carrier protein before use. Appropriate carrier proteins include but are not limited to Keyhole limpet hemacyanin (KLH). The conjugated phospho peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal (e.g., anti-14-3-3sigma) antibodies can be purified by passing the harvested antibodies through an affinity column. Monoclonal antibodies are preferred over polyclonal antibodies and can be generated according to standard methods known in the art of creating an immortal cell line which expresses the antibody.

Nonhuman antibodies are highly immunogenic in human and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal anti-14-3-3sigma antibodies.

In one embodiment, 14-3-3sigma could be included in a panel of several different antibodies, cDNA oligonucleotides, or other probes that have been demonstrated to correlate with breast cancer clinical outcome, chemotherapeutic response, and/or molecular subtypes of breast cancer, in order to improve the diagnosis, classification of molecular subtype, determination of therapeutic options, and to evaluate who will respond to chemotherapy or see no additional benefit. In such an embodiment, probes, including but not limited to antibodies or cDNA, to these targets can be immobilized on an array or a surface using conjugation techniques available to those skilled in the art, and proteins or RNA from tumor biopsies which associate with the antibodies or cDNA probes are assayed. The surface can be made of materials comprising a membrane, polystyrene, glass, metal or other surfaces. The surface can be any shape, for example, planar, spherical beads, the inner surface of a capillary, etc.

Alternatively, fresh biopsy or formalin-fixed and paraffin-embedded tissues are incubated with multiple antibodies/cDNA probes simultaneously either using a microfluidics device and/or secondary detection reagents with non-overlapping detection methods (for example, non-overlapping spectral properties, etc.), using methods known to those skilled in the art. Such a multiplex diagnostic tool would represent a significant advance in the diagnostic field, providing a resource by which tumor molecular subtype, clinical outcome, and response to treatment can be simultaneously predicted.

Additionally, 14-3-3sigma expression alone could be used to predict patient outcome, disease progression, molecular subtype, and response to treatment as shown in the examples. Kaplan-Meier analysis indicates that detection of high levels of 14-3-3sigma increased expression predicts patient outcome. Thus, the invention further provides for a method for identifying a cancer patient suitable for treatment with a 14-3-3sigma inhibitor, comprising: (a) measuring the expression level of 14-3-3sigma in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level of 14-3-3sigma indicates the patient is suitable for treatment with 14-3-3sigma inhibitor.

Thus, the present invention also provides a validated method for prognosis of basal breast cancer comprising: (a) obtaining a tissue biopsy from a patient; (b) detecting a positive level of 14-3-3sigma protein in said tissue; and (c) comparing said gene expression level to known levels of gene expression measured in a normal primary tissue of a cancer patient; whereby if said gene expression level of 14-3-3sigma is elevated as compared to said normal primary tissue, then the predictive outcome of a patient is poor.

In a preferred embodiment, the invention provides 14-3-3sigma as a biomarker of basal cancer, and more specifically basal breast cancer. Basal-like breast cancer accounts for 15-20% of breast carcinomas, and is the most aggressive of breast cancer subtypes. Unlike HER2-amplified and ER-positive subtypes, the basal subtype lacks consensus functional biomarkers that contribute to disease progression and which could be therapeutically targeted. Here, we present 14-3-3sigma as an excellent biomarker for basal-like breast cancer which correlates with tumor relapse, metastasis, and poor patient survival.

Patients with basal cancers tend to have the worst outcome. Current targeted therapeutics such as tamoxifen and Herceptin™ are available for patients having other molecular subtypes of breast cancer, such as luminal and HER2-amplified, respectively, though such targeted therapies are unavailable for basal breast cancer. Thus the present invention provides compositions and methods for detecting 14-3-3sigma as a biomarker for basal cancers to aid clinicians in providing unnecessary therapy for these patients.

In one embodiment, the 14-3-3sigma antibodies described above can be incorporated into a panel of antibodies used by pathologists to detect amplification of a set of biomarkers to inform clinicians on the type or subtype of cancer detected. A positive level of the biomarker detected by IHC, for example, would indicate the type or subtype of cancer. A positive level can be any detection level above a pre-determined threshold level in a normal tissue or sample and/or by a positive or negative control. In one embodiment, the panel of biomarkers can comprise breast cancer markers including but not limited, 14-3-3sigma, estrogen receptor (ER), progesterone (PR), EGFR, HER2, and other actin-binding or metastatic biomarkers.

For example, according to the present methods, if a cancer found to be 14-3-3-sigma (+) and for example, ER(−)/PR(−)/HER2(−), a determination can be made that the present cancer is basal breast cancer, and the clinician informed. Such a determination made early in prognosis can result in a more aggressive, different standard of care and more effective treatment provided to the patient including a lumpectomy or mastectomy followed by adjuvant administration of chemotherapeutics, ideally agents targeting 14-3-3sigma, and close monitoring for early signs of metastasis. Such a patient would not be given ER/HER2 therapy such as herceptin or tamoxifen-based therapies, which are not effective in BBC patients.

Thus in other embodiments, in other cancers, the typical standard of care can be adjusted and more aggressive therapies can be pursued immediately once a cancer has been detected as 14-3-3sigma positive (+).

In one embodiment, a method for identifying a basal cancer patient with poor prognosis, comprising: (a) measuring the expression level of the 14-3-3sigma gene in a sample from the patient; and (b) comparing the expression level of said gene from the patient with the expression level of the gene in a normal tissue sample or a reference expression level, whereby an increased expression level in said gene indicates a patient with poor prognosis.

Potential Therapeutics Targeting 14-3-3sigma-Mediated Inhibition of Actin Polymerization

In a preferred embodiment, the expression of 14-3-3sigma is modulated or manipulated. In another embodiment, treatment of premalignant amplified cells with inhibitors against 14-3-3sigma should result in the inhibition of the 14-3-3sigma gene and reduce or inhibit invasiveness of malignant cells and reduce tumor volume and/or formation. In a preferred embodiment, the compound is a 14-3-3sigma inhibitor such as a blocking or dominant-negative peptide, protein, or peptidomimetic; an antisense oligonucleotide; a siRNA oligonucleotide; a small molecule that interferes with 14-3-3sigma function; a viral vector producing a nucleic acid sequence that inhibits 14-3-3sigma; or an aptamer.

In another embodiment, 14-3-3sigma expression could be selectively targeted using RNA interference or other methods in order to decrease tumor cell invasion and eventual metastasis. In a previous example, cells were shown to have decreased migration and invasion in culture assays when deficient in 14-3-3sigma expression. To test the validity of these findings in vivo, we generated xenografts in mice using T4-2 cells having disrupted expression of 14-3-3sigma (T4-2 sh-14-3-3σ) and compared the xenografts to those formed upon injecting control cells (T4-2 sh-scrambled). It was found that tumors which formed from T4-2 sh-14-3-3σ xenografts showed a morphology characterized by more distinct margins and less perturbation to the adjacent normal tissue (resembling “carcinoma in situ”), in comparison to control tumors which are very invasive. These in vivo data indicate that disrupting 14-3-3sigma expression can inhibit tumor progression from in situ to invasive carcinoma, an important stage in the transition towards advanced, metastatic disease.

In one embodiment, 14-3-3sigma modulation can be made using optimized siRNAs. See Hannon, G. J. RNA interference (2002); Plasterk, R. H. in Science 1263-5 (2002); and Elbashir, S. M. et al. in Nature 494-8 (2001). Strong Pearson correlations between target gene amplification/expression levels and pro-apoptotic effects of siRNAs will indicate that copy number/expression levels determine the extent of apoptotic responses to target gene inhibitors.

High throughput methods can be used to identify 14-3-3sigma inhibitors such as siRNA and/or small molecular inhibitor formulations to deliver 14-3-3sigma (and other) inhibitors efficiently to cultured cells and xenografts. 14-3-3sigma (and other) inhibitory formulations will be preferentially effective against xenografts that are amplified at the target loci and that these will enhance response to platinum and taxane compounds. Effective formulations using such methods as described herein or in the examples may be developed for clinical application.

High Throughput Screening.

In one embodiment, high throughput screening (HTS) methods are used to identify compounds that inhibit or modulate 14-3-3sigma expression and/or activity. HTS methods involve providing a combinatorial chemical or peptide library containing a large number of potential therapeutic compounds (i.e., compounds that inhibit 14-3-3sigma). Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., ECIS TM, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Small Molecule or Other Inhibitors.

In one embodiment, down regulation of 14-3-3sigma at 1p36.11 will be made using inhibitors preferentially toxic to cells detected as having overamplified 14-3-3sigma. It is contemplated that such down regulation will result in loss or decrease in invasiveness of malignant cells, decrease in tumor volume and/or tumor formation.

In one embodiment, the 14-3-3sigma inhibitor is fusicoccin and/or its related family members, including but not limited to Cotylenin A, Fusicoccin A, and their metabolites, or the derivatives of above compounds designed to improve biocompatibility, bioavailability, stability, metabolism, or target specificity. These compounds stabilize the interaction between 14-3-3sigma and its ligands, thus it is predicted these compounds would deplete the cellular pool of 14-3-3sigma available to inhibit actin polymerization and thereby inhibit metastasis.

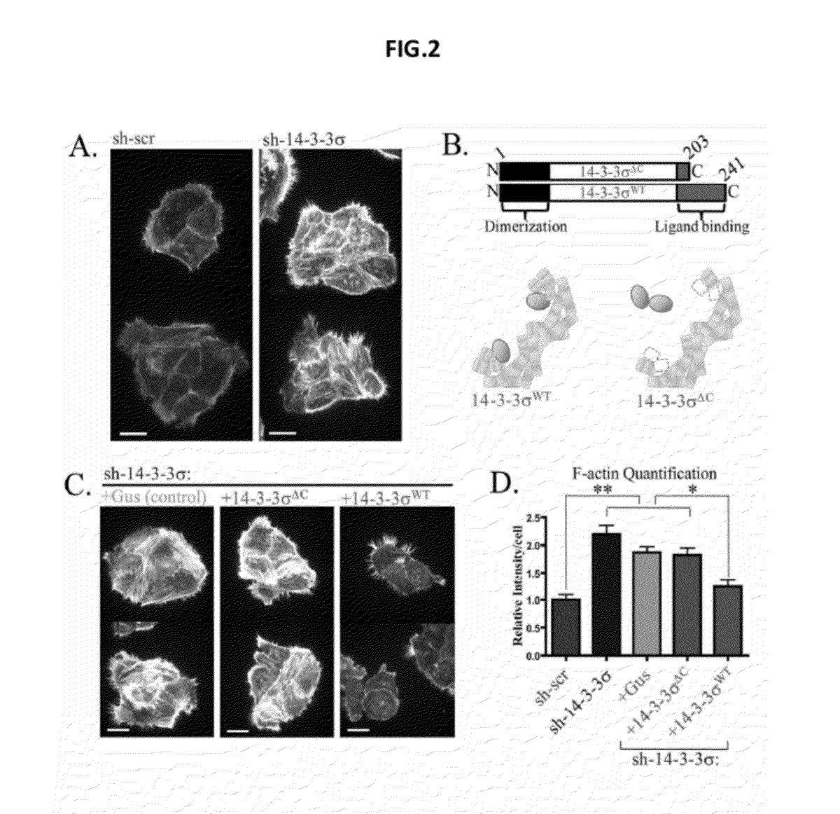

For example, Cotylenin A stabilizes the interaction between 14-3-3 and ligands at C-terminal helices shown in FIG. 2 to be involved in binding to actin. This could potentially stabilize the interaction between 14-3-3sigma and any of its numerous other ligands, essentially depleting the cellular pool of available 14-3-3sigma to interact with actin.

In another embodiment, other small molecules designed to interfere with 14-3-3-mediated interaction with actin, 14-3-3 dimerization, or to stabilize interaction between 14-3-3 and proteins other than actin, the metabolites of the above, or the derivatives of above compounds designed to improve biocompatibility, bioavailability, stability, metabolism, or target specificity. Depending on the design, these could inhibit 14-3-3::actin interaction as in the cases above.

In one embodiment, the inhibitors are agents which promote ubiquitination of 14-3-3sigma by EFP or other enzymes responsible for proteasomal degradation or the derivatives of above compounds designed to improve biocompatibility, bioavailability, stability, metabolism, or target specificity.

In another embodiment, the inhibitors are agents which promote 14-3-3 promoter methylation to silence 14-3-3 gene expression or the derivatives of above compounds designed to improve biocompatibility, bioavailability, stability, metabolism, or target specificity

Peptides and Peptidomimetics.

In another embodiment, 14-3-3sigma peptides and peptidomimetics can be used which approximate either the 14-3-3 phospholigand, the regions of actin involved in 14-3-3 interaction, or which inhibit 14-3-3sigma homodimerization, as well as their metabolites and derivatives of the above peptidomimetics that are designed to improve biocompatibility, bioavailability, stability, metabolism, target specificity, or to better approximate the three-dimensional architecture of the peptide as it exists within the 14-3-3 ligand (be it actin, 14-3-3 itself, or other 14-3-3 ligands). These peptides can be designed as inhibitors of 14-3-3 to either directly (block 14-3-3 active site to prevent interaction with actin) or indirectly (prevent 14-3-3 dimerization), and thereby rendering actin inactive or to reduce or inhibit actin polymerization.

In one embodiment, the peptides of the present invention are designed to have actin-binding activity, such that administration of the peptides results in reduced tumor growth/formation and loss of metastatic activity of basal cancer cells.

Peptidomimetics can be made using methods known in the art based on the binding region of the 14-3-3sigma protein such as SEQ ID NO:5. Peptidomimetics can be generated by using short sequences of the 14-3-3sigma protein, found at GenBank Accession Nos. CAG46724 GI:49456807; CAG46703 GI:49456765; AAH01550 GI:630737; NP_{—}006133 GI:5454052, all of which are hereby incorporated by reference. The 14-3-3sigma protein sequence can be found in GenBank Accession No: CAG46724.1 GI:49456807 (SEQ ID NO:4), which is hereby incorporated by reference.

These peptidomimetics should likely cover unique coding regions in the candidate gene. In one embodiment, the peptide comprises the amino acids 203-241 of 14-3-3sigma. Residues 203-241 of 14-3-3sigma are involved in binding actin, as a truncated version of the protein is unable to rescue the actin phenotype observed following knockdown of endogenous 14-3-3sigma (see FIG. 2). The deletion eliminated the amino acids 203-241, which comprise two helices and an acidic region known to be important for 14-3-3 interaction with ligands based on the 14-3-3sigma::ligand crystal structure. Only wild-type 14-3-3sigma was able to rescue the knockout animals showing that this region of 14-3-3sigma is crucial for 14-3-3sigma activity.

Thus in one embodiment, the peptimomimetic comprises a sequence substantially homologous to a portion of the sequence: ADLHTLSEDSYKDSTLIMQLLRDNLTLWTADNAGEEGGEAPQEPQS (SEQ ID NO:5). In another embodiment, the peptimomimetic comprises a sequence substantially identical to a portion of the sequence of SEQ ID NO:5.

The term “substantially identical” is herein used to mean having an amino acid sequence which differs only by conservative amino acid substitutions or by non-conservative amino acid substitutions, deletions, or insertions located at positions which do not destroy the biological activity of the peptide. The term “homology” or “homologous” herein when referring to an amino acid sequence similarity measured by the program, BLASTP (Altschul et al (1997), “Gapped BLAST and PSI-BLAST: a new generation of protein database search programs”, Nucleic Acids Res. 25:3389-3402 and expressed as −(% identity n/n). In measuring homology between a peptide and a protein of greater size, homology is measured only in the corresponding region; that is, the protein is regarded as only having the same general length as the peptide, allowing for gaps and insertions using default values. The term “substantially homologous” herein refers to a percent homology of at least 80%, more preferably 85%, even more preferably 90%, up to 91%, 92%, 93%, 94%, 95%, 96%, 97%, 98%, 99%, 99.5%, 99.8%, and 99.9% homology.

In another embodiment, peptidomimetics that are about 15-40 amino acids in length and substantially homologous to SEQ ID NO:5. In another embodiment, the peptidomimetic is about 15-40 amino acids in length and derived from SEQ ID NO:5. The peptides include peptides can also be designed with conservative substitutions to SEQ ID NO:5 without sacrificing activity. The term “conservative substitution” means a substitution where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr. A non-conservative amino acid substitution is one where the residues do not fall into the same class, for example, substitution of a basic amino acid for a neutral or non-polar amino acid. The invention thus further provides a method of treating a subject suffering from a disease characterized by cells having neoplastic growth, said method comprising a step of administering to the subject a therapeutically effective amount of the composition. In one aspect the subject is a human and the peptide is administered at a therapeutic dosage, for example, 0.1 to 0.5 ml, one to five times per week.

The present peptides may be formulated according to known pharmaceutical technology. They may be administered singly or in combination, and may further be administered in combination with other cancer or actin-binding drugs. They may be conventionally prepared with excipients and stabilizers in sterilized, lyophilized powdered form for injection, or prepared with stabilizers and peptidase inhibitors of oral and gastrointestinal metabolism for oral administration. Alternatively, the peptides can be prepared with additives or fused to carrier molecules that would increase peptide efficacy and cell entry. Peptides containing naturally occurring amino acids may be produced intracellularly by introduction of DNA or RNA constructs.

Inhibitor Oligonucleotide and RNA Interference (RNAi) Sequence Design.

In one embodiment, known methods are used to identify sequences that inhibit 14-3-3sigma and its role in cell acquisition of invasiveness. Such inhibitors may include but are not limited to, siRNA oligonucleotides, antisense oligonucleotides, peptide inhibitors and aptamer sequences that bind and act to inhibit 14-3-3sigma gene expression and/or function.

In one embodiment, RNA interference is used to generate small double-stranded RNA (small interference RNA or siRNA) inhibitors to affect the expression of a candidate gene generally through cleaving and destroying its cognate RNA. Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu F Y. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth J, Elbashir S M, Vandenburgh K, Manning a H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall W S, Khvorova A. (2004) Rational siRNA design for RNA interference. Nat Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (URL:<http://www.idtdna.com/SciTools/SciTools.aspx>), or siDESIGN™ Center from Dharmacon, Inc. (URL:<http://design.dharmacon.com/default.aspx?source=0>). siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 3′ or 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In a preferred embodiment, the 14-3-3sigma siRNA oligonucleotide targets the 14-3-3sigma sequence GTG ACC ATG TTT CCT CTC A (SEQ ID NO: 6) and is labeled with Alexa 488 at the 3′ end. In a preferred embodiment, the 14-3-3sigma siRNA oligonucleotide sequences are

| (SEQ ID NO: 7) | |
|---|---|
| Sense: | GTG ACC ATG TTT CCT CTC A |
| (SEQ ID NO: 8) | |
| Antisense: | TGA GAG GAA ACA TGG TCA C |

In another embodiment, antisense oligonucleotides (“antisense oligos”) can be designed to inhibit 14-3-3sigma and other candidate gene function. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the 14-3-3sigma and other candidate gene antisense oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA sequence (e.g. 14-3-3sigma target mRNA sequence SEQ ID NO: 4) will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

In another embodiment, aptamer sequences which bind to specific RNA or DNA sequences can be made. Aptamer sequences can be isolated through methods such as those disclosed in co-pending U.S. Patent Appl. Pub No. US-2009-0075834-A1, entitled, “Aptamers and Methods for their In vitro Selection and Uses Thereof,” which is hereby incorporated by reference.

Aptamers (DNA, peptide-based, or otherwise) structurally mimicking the regions of actin that either bind to 14-3-3 ligand interaction site, inhibit 14-3-3 dimerization, or stabilize the interaction of 14-3-3 with proteins other than actin, the metabolites of the above, or the derivatives of above compounds designed to improve biocompatibility, bioavailability, stability, metabolism, or target specificity. Depending on the design, these could inhibit 14-3-3::actin interaction as described above.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E. W. & Lipman, D. J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

Recombinant Expression.

14-3-3sigma inhibitors such as the siRNA 14-3-3sigma inhibitor described herein can also be expressed recombinantly. In general, the nucleic acid sequences encoding 14-3-3sigma inhibitors such as the siRNA 14-3-3sigma inhibitor and related nucleic acid sequence homologues can be cloned. This aspect of the invention relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)).

14-3-3sigma can be first cloned from cDNA and genomic DNA libraries or isolated using amplification techniques with oligonucleotide primers. For example, sequences of candidate genes are typically isolated from nucleic acid (genomic or cDNA) libraries by hybridizing with a nucleic acid probe, the sequence of which can be derived from publicly available genomic sequence. In another embodiment, RNA and genomic DNA can be isolated from any mammal including: primates such as humans, monkeys, and chimpanzees; rodents, including mice and rats. Methods for making and screening cDNA libraries and genomic DNA libraries are well known (see, e.g., Gubler & Hoffman, Gene 25:263-269 (1983); Sambrook et al., supra; Ausubel et al., supra; Benton & Davis, Science 196:180-182 (1977); and Grunstein et al., PNAS USA, 72:3961-3965 (1975)).

Nucleic acids encoding 14-3-3sigma can also be isolated from expression libraries using antibodies as probes. Such polyclonal or monoclonal antibodies can be raised using, for example, the polypeptides comprising the sequences such as the 14-3-3sigma protein sequence set forth in GenBank Accession No. NP_{—}006133, and subsequences thereof, using methods known in the art (see, e.g., Harlow and Lane, Antibodies: A Laboratory Manual (1988)).

Substantially identical nucleic acids encoding sequences of the candidate genes can be isolated using nucleic acid probes and oligonucleotides under stringent hybridization conditions, by screening libraries.

Alternatively, expression libraries can be used to clone these sequences, by detecting expressed homologues immunologically with antisera or purified antibodies made against the core domain of nucleic acids encoding sequences of the candidate genes which also recognize and selectively bind to the homologue.

Gene expression of the candidate genes amplified and found to be causal in acquisition of invasiveness, such as 14-3-3sigma, can also be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like.

To obtain high level expression of a cloned gene or nucleic acid sequence, such as those cDNAs encoding nucleic acid sequences encoding 14-3-3sigma inhibitors such as the siRNA 14-3-3sigma inhibitor and related nucleic acid sequence homologues, one typically subclones an inhibitor peptide sequence (e.g., nucleic acid sequences encoding 14-3-3sigma inhibitors such as a siRNA 14-3-3sigma sequence encoding SEQ ID NOS: 7-8) into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding 14-3-3sigma inhibitors such as the siRNA 14-3-3sigma inhibitor or a subsequence thereof. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant 14-3-3sigma inhibitors peptides to provide convenient methods of isolation, e.g., His tags. In some case, enzymatic cleavage sequences (e.g., Met-(His)g-Ile-Glu-GLy-Arg which form the Factor Xa cleavage site) are added to the recombinant 14-3-3sigma inhibitor peptides. Bacterial expression systems for expressing the 14-3-3sigma inhibitor peptides and nucleic acids are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of 14-3-3sigma inhibitor, which can then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al., eds, 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing 14-3-3sigma inhibitor peptides and nucleic acids.

After the expression vector is introduced into the cells, the transfected cells are cultured under conditions favoring expression of 14-3-3sigma inhibitors such as the siRNA 14-3-3sigma inhibitor and related nucleic acid sequence homologues.

Methods of Treatment.

The 14-3-3sigma inhibitor peptides and nucleic acids of the present invention can be used to reduce or prohibit tumor volume and/or formation, and inhibit invasiveness of cancers, especially those found in basal cancers. The peptides and nucleic acids are administered to a patient in an amount sufficient to elicit a therapeutic response in the patient (e.g., reduction of tumor size and growth rate, prolonged survival rate, reduction in concurrent cancer therapeutics administered to patient, inhibition of metastasis or invasiveness). An amount adequate to accomplish this is defined as “therapeutically effective dose or amount.”

The peptides and nucleic acids of the invention can be administered directly to a mammalian subject using any route known in the art, including e.g., by injection (e.g., intravenous, intraperitoneal, subcutaneous, intramuscular, intratumoral or intradermal), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the peptides and nucleic acids of the invention can be in any convenient manner, e.g., by injection, intratumoral injection, intravenous and arterial stents (including eluting stents), cather, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17th ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., ovarian cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, polypeptides and nucleic acids of the present invention can be administered at a rate determined by the LD-50 of the polypeptide or nucleic acid, and the side-effects of the polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the 14-3-3sigma inhibitor peptides and nucleic acids disclosed herein parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or siSTABLE siRNAs are readily soluble in aqueous solution and can be resuspended at concentrations as high as 2.0 mM. However, viscosity of the resultant solutions can sometimes affect the handling of such concentrated solutions.

While lipid formulations have been used extensively for cell culture experiments, the attributes for optimal uptake in cell culture do not match those useful in animals. The principle issue is that the cationic nature of the lipids used in cell culture leads to aggregation when used in animals and results in serum clearance and lung accumulation. Polyethylene glycol complexed-liposome formulations are currently under investigation for delivery of siRNA by several academic and industrial investigators but typically require complex and formulation knowledge. There are a few reports that cite limited success using lipid-mediated delivery of plasmids or oligonucleotides in animals.

Oligonucleotides can also be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

Liposomes.

In certain embodiments, the inventors contemplate the use of liposomes, nanocapsules, microparticles, microspheres, lipid particles, vesicles, and the like, for the administration of the 14-3-3sigma inhibitory peptides and nucleic acids of the present invention. In particular, the compositions of the present invention may be formulated for delivery either encapsulated in a lipid particle, a liposome, a vesicle, a nanosphere, or a nanoparticle or the like. In one embodiment, the 14-3-3sigma siRNA inhibitors (e.g., SEQ ID NOS: 7-8) are entrapped in a liposome for delivery.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. Liposomes are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types. Alternatively, the invention provides for pharmaceutically-acceptable nanocapsule formulations of the compositions of the present invention.

Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684).

Gene Therapy.

In certain embodiments, the nucleic acids encoding inhibitory 14-3-3sigma peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses an inhibitory 14-3-3sigma peptides and nucleic acids of the present invention, thereby mitigating the effects of over amplification of a candidate gene associated with reduced survival rate.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Bohm eds., 1995); and Yu et al., Gene Therapy 1:13-26 (1994)).

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as described in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al. (1988) Mol. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875). Additional suitable vectors include E1B gene-attenuated replicating adenoviruses described in, e.g., Kim et al., Cancer Gene Ther. 9(9):725-36 (2002) and nonreplicating adenovirus vectors described in e.g., Pascual et al., J. Immunol. 160(9):4465-72 (1998) Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory 14-3-3sigma nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

Other known viral-based delivery systems are described in, e.g., Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; Guzman et al. (1993) Cir. Res. 73:1202-1207; and Lotze and Kost, Cancer Gene Ther. 9(8):692-9 (2002).

Combination Therapy.

In some embodiments, the inhibitory 14-3-3sigma polypeptides and nucleic acids are administered in combination with a second therapeutic agent for treating or preventing cancer, including ovarian, breast, colon, and other cancers. For example, an inhibitory 14-3-3sigma siRNA of SEQ ID NO: 5-6 may be administered in conjunction with any of the standard treatments for cancer including, but not limited to, paclitaxel, cisplatin, carboplatin, chemotherapy, and radiation treatment.

The inhibitory 14-3-3sigma polypeptides and nucleic acids and the second therapeutic agent may be administered simultaneously or sequentially. For example, the inhibitory 14-3-3sigma polypeptides and nucleic acids may be administered first, followed by the second therapeutic agent. Alternatively, the second therapeutic agent may be administered first, followed by the inhibitory 14-3-3sigma polypeptides and nucleic acids. In some cases, the inhibitory 14-3-3sigma polypeptides and nucleic acids and the second therapeutic agent are administered in the same formulation. In other cases the inhibitory 14-3-3sigma polypeptides and nucleic acids and the second therapeutic agent are administered in different formulations. When the inhibitory 14-3-3sigma polypeptides and nucleic acids and the second therapeutic agent are administered in different formulations, their administration may be simultaneous or sequential.

In some cases, the inhibitory 14-3-3sigma polypeptides and nucleic acids can be used to target therapeutic agents to cells and tissues expressing 14-3-3sigma and other candidate genes that are related to acquisition of invasiveness in premalignant cells.

Kits.

The present invention further provides kits for use within any of the above diagnostic methods. Such kits typically comprise two or more components necessary for performing a diagnostic assay. Components may be compounds, reagents, containers and/or equipment. For example, one container within a kit may contain an inhibitory 14-3-3sigma polypeptides and nucleic acids. One or more additional containers may enclose elements, such as reagents or buffers, to be used in the assay. Such kits may also, or alternatively, contain a detection reagent as described above that contains a reporter group suitable for direct or indirect detection of antibody binding.

Kits can also be supplied for therapeutic uses. Thus, the subject composition of the present invention may be provided, usually in a lyophilized form, in a container. The inhibitory 14-3-3sigma polypeptides and nucleic acids described herein are included in the kits with instructions for use, and optionally with buffers, stabilizers, biocides, and inert proteins. Generally, these optional materials will be present at less than about 5% by weight, based on the amount of polypeptide or nucleic acid, and will usually be present in a total amount of at least about 0.001% by weight, based on the polypeptide or nucleic acid concentration. It may be desirable to include an inert extender or excipient to dilute the active ingredients, where the excipient may be present in from about 1 to 99% weight of the total composition. The kits may further comprise a second therapeutic agent, e.g., paclitaxel, carboplatin, a chemotherapeutic agent.

### Example 1

### 14-3-3sigma Correlates with Malignant Progression in Two BBC Tumor Models

In the present study, we discovered a novel function for 14-3-3sigma in coordinating cell invasiveness and motility within both the HMT-3522 and the MCF10 progression series through directly regulating actin dynamics downstream of canonical cytoskeletal signaling pathways. 14-3-3sigma directly inhibits actin polymerization in vitro and in situ to define the boundaries of microfilament architecture within cells, and in migratory cells, 14-3-3sigma polarizes away from the leading edge to allow the dynamic actin remodeling necessary for directional migration. In a tissue microarray (TMA) spotted with tumor biopsies from 245 cases of invasive breast carcinoma, we show 14-3-3sigma is a BBC histological biomarker with high sensitivity (70%; 16/23 basal tumors positive) and specificity (91%; 15/164 non-basal tumors positive), and when used in conjunction with ER negativity, 14-3-3sigma positivity provides a predictive prognostic value of 94% (<6% false positives) for correctly identifying BBC cases. Correspondingly, 14-3-3sigma immunoreactivity correlates with lower 5-year metastasis-free and recurrence-free survival within the TMA cohort, and 14-3-3sigma expression similarly correlates with tumor relapse, metastasis, and poor survival in two independent patient cohorts. We argue that rather than being a breast tumor suppressor in the basal subtype, 14-3-3sigma represents the first “functional biomarker” of BBC which may contribute directly to tumor dissemination by modulating actin dynamics, making 14-3-3sigma a promising target of therapeutic intervention for patients with BBC.

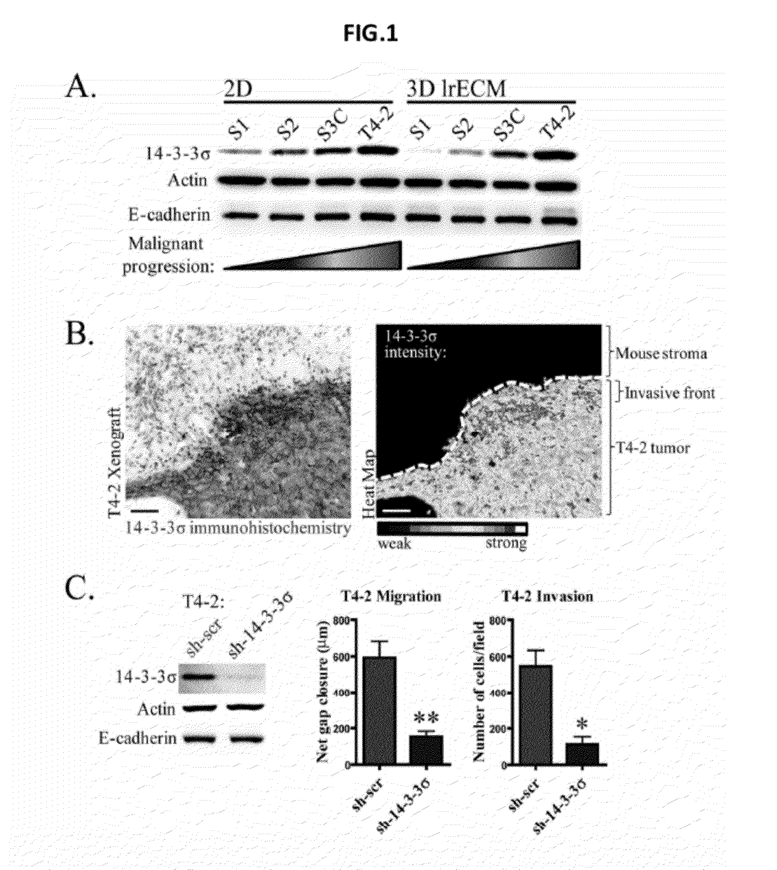

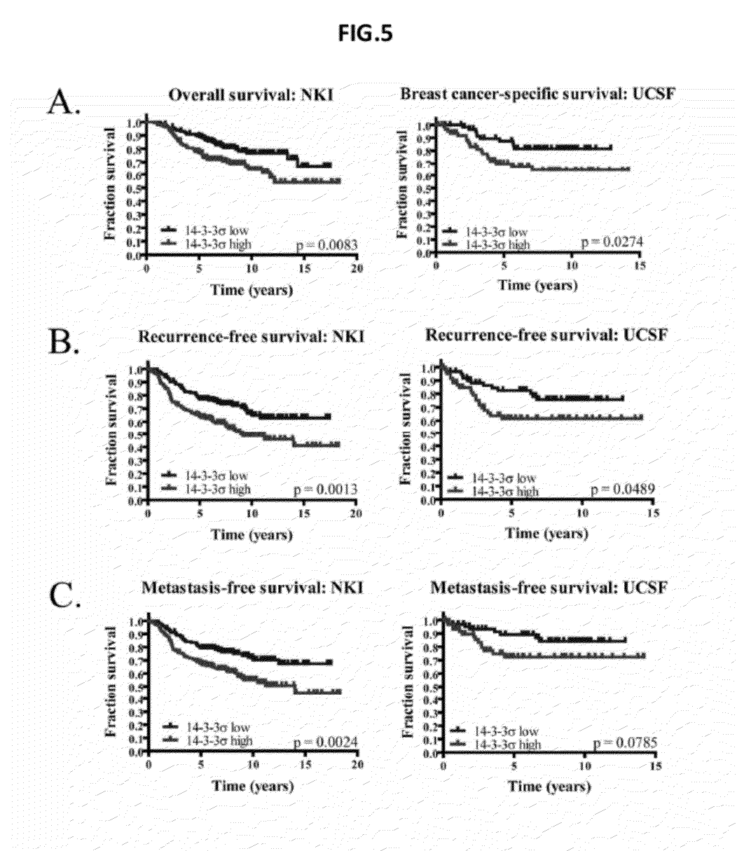

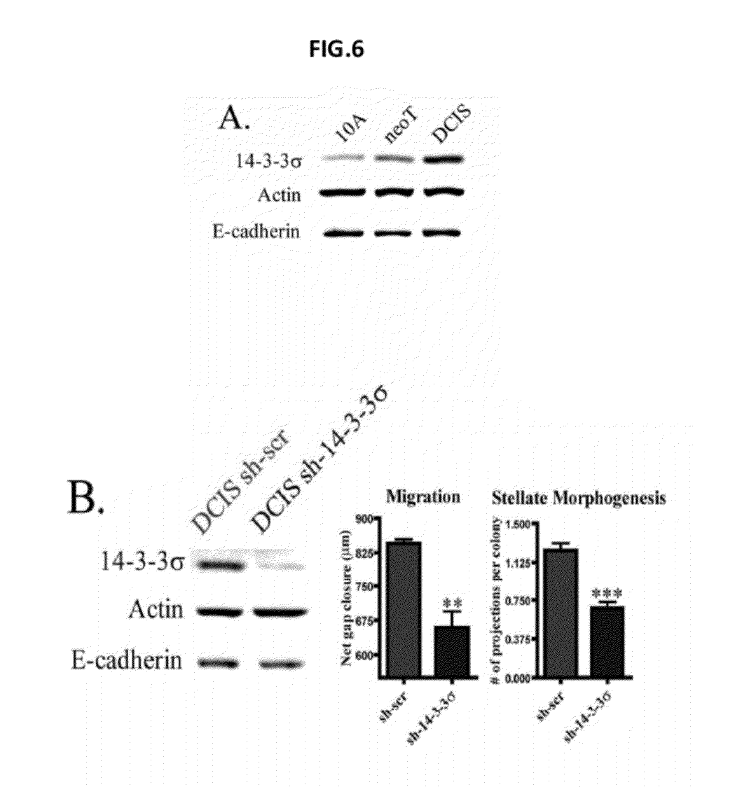

We performed proteomic profiling of the HMT-3522 progression series (S1 and T4-2) to attempt to identify and validate novel biomarkers of BBC, and to elucidate what function (if any) they serve during malignant progression. In this screen, we identified 14-3-3sigma as being over 2-fold elevated in the tumorigenic T4 cells relative to their non-tumorigenic counterpart S1 cells when grown in 3D lrECM (Table 1). Of the proteins discovered, we selected 14-3-3sigma for further analysis because it was independently shown to be higher in T4-2 versus S1 cells [14] and it is a histological marker of breast myoepithelial cells [25, 26], suggesting it may have a novel function related to BBC tumor progression. By western blot analysis, 14-3-3sigma correlates well with HMT-3522 progression from non-malignant (S1), to pre-malignant and EGF independent (S2), to pre-invasive and highly proliferative (S3C), and to robust tumorigenicity and invasiveness (T4-2) (FIG. 1). Similarly, in the MCF-10 progression series, 14-3-3sigma expression increases as cells become more aggressive from non-malignant (MCF10A), to premalignant and hyperproliferative (MCF10neoT), to forming comedo-type DCIS in vivo (MCF10DCIS.com) (FIG. 6A), indicating that 14-3-3sigma expression correlates well with tumorigenesis in two independent models of BBC progression.

Strong 14-3-3sigma immunoreactivity was observed throughout T4-2 tumors, with intensification occurring at the invasive front where soft tissue infiltration occurs (FIG. 1B), suggesting that 14-3-3sigma may promote tumor invasion into the surrounding mouse stroma. To address whether 14-3-3sigma contributes to T4-2 invasion and migration, cell lines stably expressing shRNAs targeting 14-3-3sigma or a non-specific sequence were generated. Reduced 14-3-3sigma expression does not influence T4-2 cell proliferation or cell cycle distribution (data not shown), but rather decreases their motility in scratch assays, and their invasion through lrECM-coated transwell inserts (FIG. 1C). Likewise, MCF10 DCIS.com cells expressing shRNA hairpins targeting 14-3-3sigma have decreased motility (FIG. 6B), though relative to T4-2, even parental MCF10DCIS.com cells are not invasive through lrECM-coated transwell inserts in agreement with their ability to form highly proliferative but non-invasive comedo DCIS at low passages in mouse xenografts [13, 27, 28]. However, MCF10DCIS.com cells with reduced 14-3-3sigma expression form colonies in 3D lrECM with fewer migratory and invasive projections per colony (decreased stellate morphogenesis) than control cells expressing scrambled shRNA hairpins (FIG. 6B). We argue that stellate colony morphogenesis correlates well with aggressive cell behavior and invasive properties [29], suggesting the MCF10DCIS.com cells deficient in 14-3-3sigma have less invasive potential. Taken together, these data indicate a role for 14-3-3sigma in regulating the migratory and invasive phenotype of aggressive cell lines in two independent, isogenic models of breast tumor progression with basal-like features.

### Example 2

### 14-3-3sigma Inhibits Actin Polymerization Downstream of Canonical Signaling

Cell migration requires dynamic remodeling of the actin cytoskeleton at both the leading and trailing edge of the cell in response to biochemical and/or mechanical stimuli [30-32]. To address whether the perturbed migration observed in cells with reduced expression of 14-3-3sigma is coupled with changes to the actin cytoskeletal architecture, cells were stained with phalloidin to decorate F-actin and imaged by confocal microscopy. In agreement with the observed migratory deficiency, T4-2 cells with deficient 14-3-3sigma have a greater than 2-fold increase in actin polymerization, particularly an increase in actin stress fiber formation, relative to T4-2 cells expressing nonspecific shRNA hairpins (FIG. 2A).

To address whether 14-3-3sigma inhibits actin polymerization in a ligand binding mechanism and to eliminate the possibility that the actin phenotype is an off-target effect of RNA interference, constructs were generated (FIG. 2B) that enable forced expression of wild-type 14-3-3sigma (14-3-3σ^{WT}) or a C-terminal truncated 14-3-3sigma (14-3-3σ^{ΔC}). 14-3-3σ^{ΔC} is the human equivalent of the 14-3-3sigma mutation responsible for the repeated-epilation mouse phenotype [33, 34], and the truncation eliminates two helixes and an acidic region thought to be important for ligand binding [35-37]. When introduced into 14-3-3sigma knockdown cells, only 14-3-3σ^{WT} was able to rescue the deregulated actin polymerization in comparison to an unrelated gene (Gus) and to 14-3-3σ^{ΔC} (FIG. 2B,C). Taken together, these data indicate that 14-3-3sigma inhibits actin polymerization in cells, that this occurs in a ligand binding mechanism, and that the actin phenotype is not an artifact of shRNA expression.

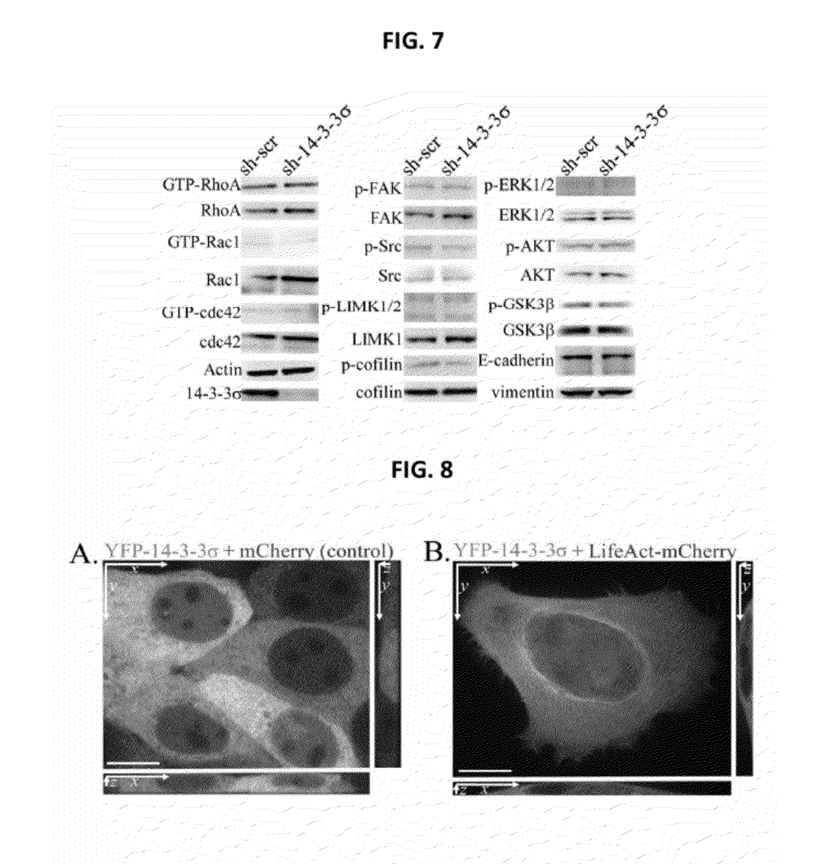

Given that the actin phenotype depends on ligand interaction, we attempted to unravel the signaling events regulated by 14-3-3sigma which are perturbed following RNA interference, and considered canonical migration and cytoskeletal signaling as a starting point. During cell migration, stimuli from the cell microenvironment drive PI3K-PIP3-dependent localized activation of Rho family GTPases (Rac, Rho, and cdc42) which, depending on the stoichiometry of their activity and the activity of other factors such as cofilin, Src, Erk, and LIMK among others, can stimulate formation of discreet actin structures which drive polarized, directional cell migration [30, 31, 38-41]. By western blot analysis, we show that 14-3-3sigma knockdown does not influence cytoskeletal signaling through these signaling molecules, nor through epithelial to mesenchymal transition (FIG. 7), indicating that 14-3-3sigma inhibits actin polymerization in a ligand-binding manner and downstream of canonical signaling pathways. Given that an important prerequisite to motility is the ability of cells to dramatically reorganize their actin cytoskeleton, loss of the spatiotemporal regulation of actin polymerization in cells with decreased 14-3-3sigma is in agreement with their motility phenotype (FIG. 1, S1).

### Example 3

### 14-3-3sigma Interacts Directly with Actin to Inhibit its Polymerization

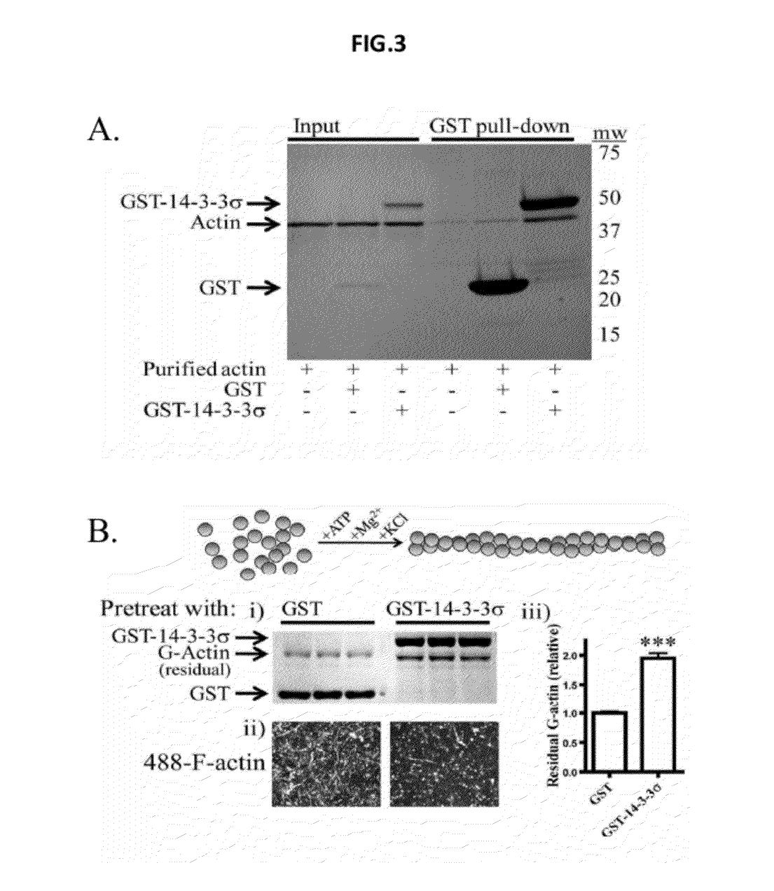

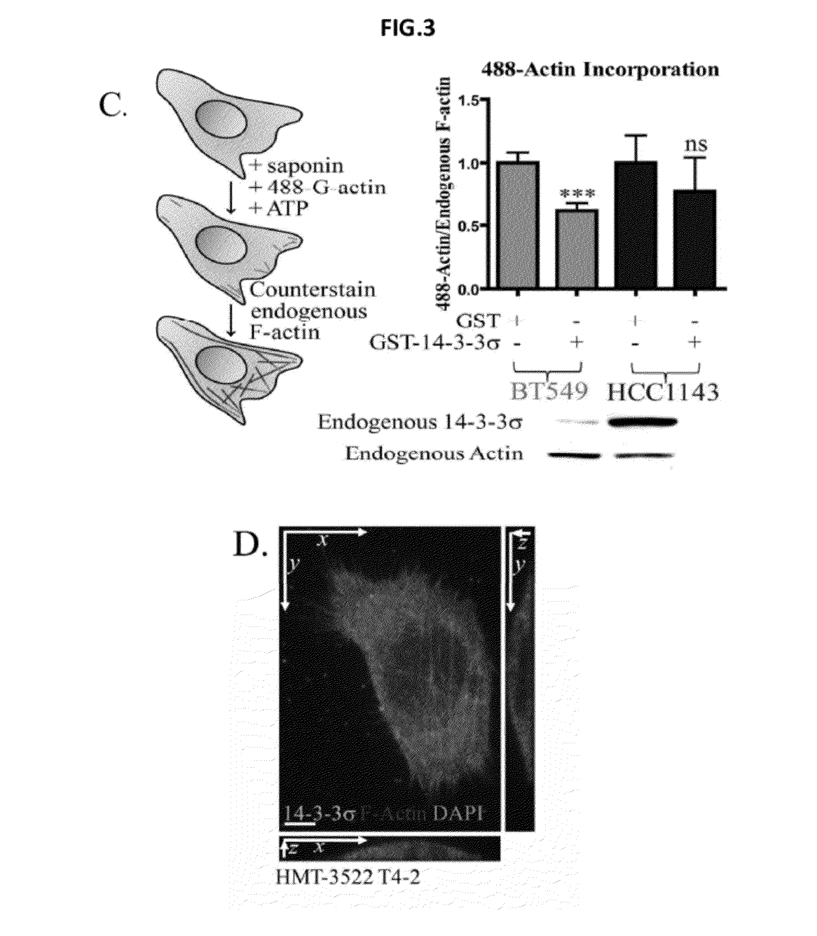

As 14-3-3sigma regulates actin structure downstream of small GTPases and other actin reorganizing factors, and 14-3-3sigma is known by mass spectrometry-based analysis to interact with numerous ligands (including actin) (Wilker E W, van Vugt M A, Artim S A, Huang P H, Petersen C P, Reinhardt H C, Feng Y, Sharp P A, Sonenberg N, White F M et al: 14-3-3sigma controls mitotic translation to facilitate cytokinesis. 2007 Nature, 446(7133):329-332), we asked whether recombinant 14-3-3sigma would interact with purified actin in vitro and whether this mechanism is responsible for the actin phenotype observed. In a GST-pull down experiment, we demonstrate that GST-14-3-3sigma, but not GST alone, is able to interact with actin in solution (FIG. 3A). To test whether this interaction inhibits actin polymerization, G-actin was pretreated with an equimolar ratio of either GST or GST-14-3-3, then polymerized into F-actin by adding KCl, Mg²⁺, and ATP (FIG. 3B). Pretreating G-actin with GST-14-3-3sigma significantly attenuates actin polymerization, as shown by a ˜2-fold retention in the amount of soluble G-actin following polymerization relative to pretreatment with GST alone. A similar effect was observed using AlexaFluor488-conjugated actin (488-actin), where fewer actin fibers were visible following 488-G-actin polymerization when pretreated with GST-14-3-3sigma. These data demonstrate that 14-3-3sigma interacts directly with actin in vitro, and that 14-3-3sigma interaction is sufficient to inhibit the polymerization of G-actin to F-actin in the absence of cofactors.

To test whether 14-3-3sigma antagonizes actin polymerization in situ rather than exclusively with purified components, 488-G-actin pretreated with either GST or GST-14-3-3sigma was introduced into cells that were gently permeabilized, and the ratio of actin incorporation into the endogenous F-actin cytoskeleton was measured by counterstaining fixed cells with phalloidin (FIG. 3C). In cells having very low endogenous 14-3-3sigma (BT549), 488-G-actin pretreated with GST-14-3-3sigma was incorporated significantly less into the endogenous actin cytoskeleton than when pretreated with GST alone. However, this was not observed in cells having high endogenous 14-3-3sigma (HCC1143), suggesting the endogenous 14-3-3sigma is sufficient to inhibit 488-G-actin incorporation in these cells. Taken together, these in vitro and in situ experiments (FIG. 3A-C) provide evidence supporting a previously unknown mechanism through which 14-3-3sigma regulates actin dynamics by directly associating with actin and inhibiting its polymerization. This inhibition leads to a mutually-exclusive distribution of 14-3-3sigma and F-actin in cells (FIG. 3D), suggesting that 14-3-3sigma defines the boundary where actin polymerization occurs, and when 14-3-3sigma is depleted from cells, the tightly regulated actin polymerization is no longer observed.

### Example 4

### 14-3-3sigma Polarizes away from the Leading Edge of a Cell During Migration

During chemotaxis, cells polarize such that factors involved in the formation of actin-dependent protrusions localize to the leading edge of the cell, whereas factors involved in actinomyosin-based contractility or which antagonize components involved in formation of actin-based protrusions localize to the trailing edge of a cell to enforce directional migration. Several lines of evidence presented in the above examples suggested that 14-3-3sigma binds to and directly inhibits actin polymerization, leading us to speculate that its subcellular distribution may dictate where actin polymerization occurs in live cells, and that 14-3-3sigma may polarize away from the migratory front where dynamic actin remodeling is necessary during directional cell migration. To follow protein trafficking during cell migration, genetically-encoded probes were developed to monitor the subcellular distribution of 14-3-3sigma (YFP-14-3-3σ) and F-actin (Lifeact-mCherry) in living cells. Lifeact is a 17-mer peptide from the actin-binding domain of S. cerevisiae Abp 140, which preferentially decorates F-actin to act much like a live-cell phalloidin. In T4-2 cells weakly expressing each fluorophore (to minimize the chance of disrupting endogenous F-actin dynamics), the subcellular distribution of YFP-14-3-3sigma and Lifeact-mCherry is mutually-exclusive (FIG. 8) similar to the distribution of endogenous 14-3-3sigma and F-actin (FIG. 3D), providing additional evidence to suggest that 14-3-3sigma inhibits actin polymerization in cells, and validates the live-cell fluorescent probes approximate their endogenous counterparts with respect to cellular distribution.

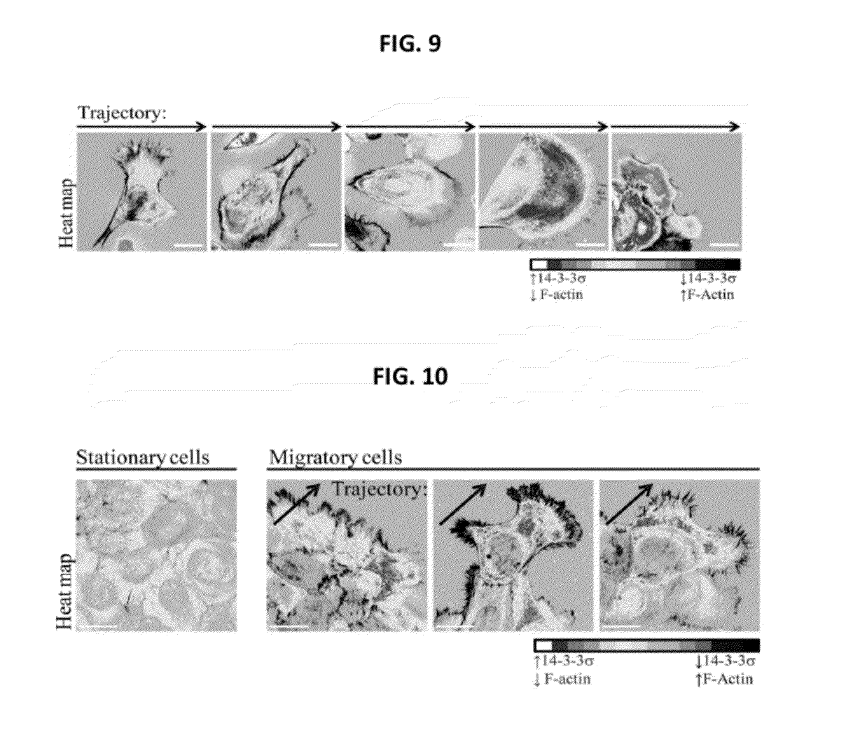

Using a spinning-disc confocal microscope fitted with a cell incubator, we were able to visualize 14-3-3sigma and F-actin distribution in living cells for time intervals exceeding 24 hours. We found that during cell migration, 14-3-3sigma polarizes away from the leading edge of a cell where the most dynamic F-actin remodeling occurs (FIG. 4), and that cells actively migrating 24 hours later are still polarized (FIG. 9), indicating that this gradient persists throughout migration. Similarly, endogenous 14-3-3sigma polarizes away from the F-actin-rich leading edge in parental T4-2 cells during migration, but no polarization occurs in stationary cells (FIG. 10). Taken together, these experiments provide evidence that 14-3-3sigma defines regions of actin polymerization to coordinate cytoskeletal homoeostasis and migration, and that similar to many actin regulatory proteins, 14-3-3sigma itself becomes polarized during motility to facilitate directional cell migration.

### Example 5

### 14-3-3sigma is a Novel BBC Biomarker which Correlates with Poor Prognosis

Our data to this point suggest that 14-3-3sigma expression correlates with acquisition of aggressive cellular behavior in two BBC tumor progression models, and that 14-3-3sigma regulates actin dynamics in cells to coordinate migratory and invasive characteristics. We next evaluated whether 14-3-3sigma expression correlates with BBC in vivo and with poor patient prognosis by performing immunohistochemical staining on a tissue microarray spotted with tumor cores from 245 patients, which have been uniformly treated with adjuvant anthracycline-based chemotherapy following surgery, and which have been extensively characterized by expression of breast cancer histological markers in prior studies (Thorat M A, Marchio C, Morimiya A, Savage K, Nakshatri H, Reis-Filho J S, Badve S: Forkhead box A1 expression in breast cancer i s associated with luminal subtype and good prognosis. 2008 J Clin Pathol, 61(3):327-332; Tan D S, Marchio C, Jones R L, Savage K, Smith I E, Dowsett M, Reis-Filho J S: Triple negative breast cancer: molecular profiling and prognostic impact in adjuvant anthracycline-treated patients. 2008 Breast Cancer Res Treat, 111(1):27-44; Savage K, Leung S, Todd S K, Brown L A, Jones R L, Robertson D, James M, Parry S, Rodrigues Pinilla S M, Huntsman D et al: Distribution and significance of caveolin 2 expression in normal breast and invasive breast cancer: an immunofluorescence and immunohistochemical analysis. 2008 Breast Cancer Res Treat, 110(2):245-256; Arriola E, Rodriguez-Pinilla S M, Lambros M B, Jones R L, James M, Savage K, Smith I E, Dowsett M, Reis-Filho J S: Topoisomerase II alpha amplification may predict benefit from adjuvant anthracyclines in HER2 positive early breast cancer. 2007 Breast Cancer Res Treat, 106(2):181-189; Savage K, Lambros M B, Robertson D, Jones R L, Jones C, Mackay A, James M, Hornick J L, Pereira E M, Milanezi F et al: Caveolin 1 is overexpressed and amplified in a subset of basal-like and metaplastic breast carcinomas: a morphologic, ultrastructural, immunohistochemical, and in situ hybridization analysis. 2007 Clin Cancer Res, 13(1):90-101; Rodriguez-Pinilla S M, Jones R L, Lambros M B, Arriola E, Savage K, James M, Pinder S E, Reis-Filho J S: MYC amplification in breast cancer: a chromogenic in situ hybridisation study. 2007 J Clin Pathol, 60(9):1017-1023; and Reis-Filho J S, Savage K, Lambros M B, James M, Steele D, Jones R L, Dowsett M: Cyclin D1 protein overexpression and CCND1 amplification in breast carcinomas: an immunohistochemical and chromogenic in situ hybridisation analysis. 2006 Mod Pathol, 19(7):999-1009).

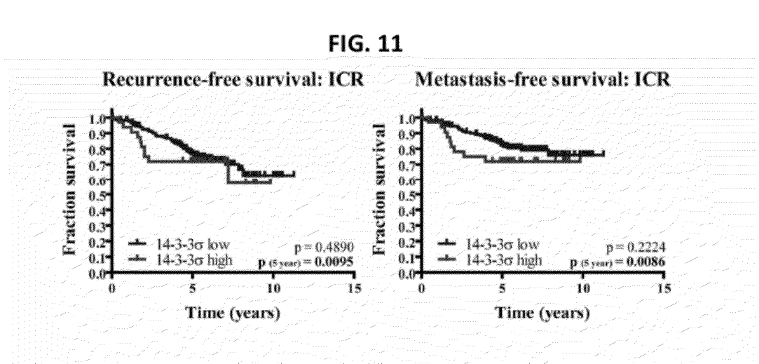

Strong 14-3-3sigma cytoplasmic expression comparable to normal breast myoepithelial cell staining intensity was observed in 16.2% of cases (Table 1), a prevalence similar to that previously reported (18.9%) in an independent cohort of patients using the same immunohistochemical method (Simpson, et al. 2004 J Pathol, 202(3):274-285). 14-3-3sigma immunoreactivity positively correlated with high histological grade, with high Ki-67 proliferative index, and with expression of several BBC markers (Ck 5/6, Ck 14, Ck 17, EGFR, and caveolins 1 and 2), and inversely correlated with luminal tumor markers (ER, PR, and FOXA1). When tumors were subclassified into luminal, basal, and HER2-amplified subtypes using the immunohistochemical surrogate of Nielsen and colleagues (Immunohistochemical and clinical characterization of the basal-like subtype of invasive breast carcinoma. 2004 Clin Cancer Res, 10(16):5367-5374), strong 14-3-3sigma immunoreactivity was observed in 70% (16/23) of BBC tumors and in only 9% (15/164) of non-basal tumors. When used in conjunction with ER-negativity, 14-3-3sigma positivity provided a predictive prognostic value of 94% (1/17 false positive rate; Fisher's exact test p value<0.0001) for correctly identifying basal tumors. These data provide strong evidence that 14-3-3sigma is a sensitive and specific novel BBC biomarker in ER-negative tumors. Correspondingly, 14-3-3sigma immunoreactivity correlated with decreased 5-year recurrence-free and metastasis-free survival in this cohort (FIG. 11).

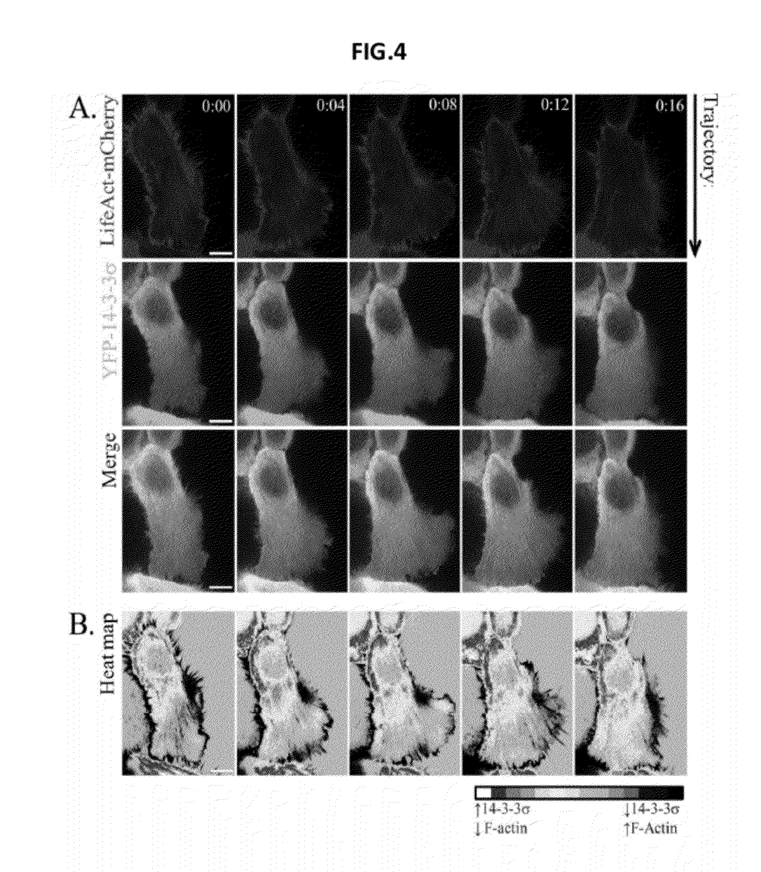

To address whether 14-3-3sigma contributes to patient outcome in independent studies, we interrogated gene expression profiles of two breast cancer patient cohorts with well-characterized disease progression and clinical follow-up [56-58]. In the Netherlands 295-patient (NKI) cohort, 14-3-3sigma expression correlates with poorer breast cancer-specific survival and shorter time to breast cancer recurrence and to metastasis (FIG. 5). A similar trend was observed in the San Francisco 130-patient (UCSF) cohort, though it should be noted that the UCSF cohort had significantly longer metastasis-free survival than the NKI cohort (data not shown), which in conjunction with the smaller cohort size, may explain the lower statistical significance. Taken together we argue that in BBC, 14-3-3sigma does not function as a breast cancer tumor suppressor, but rather may contribute to tumor progression to metastatic disease by regulating actin dynamics.

### Example 6

### Experimental Procedures Used in the Examples

Cell Culture. HMT-3522 S1, S2, S3C, and T4-2 were maintained in tissue culture monolayers or in 3D cultures on lrECM (Matrigel™) as previously described [3, 4, 7, 90]. MCF10neoT and MCF10DCIS.com cells were obtained from Dr. Fred Miller (Karmanos Cancer Institute, Detroit, Mich.) and cultured in the same manner as MCF10A cells [91], which were available from ATCC. BT549 and HCC1143 cell lines were obtained from ATCC and were maintained in RPMI-1640 media supplemented with 10% FBS and penicillin/streptomycin [92].

Western Blot and Small GTPase Assays:

RIPA buffer cell lysates were resolved using precast 4-20% gradient SDS-PAGE gels (Invitrogen), then transferred onto 0.45 μm nitrocellulose membrane (Whatman) for western blot analysis using antibodies targeting 14-3-3sigma (C18; Santa Cruz), actin ([clone AC-15]; Sigma-Aldrich), and E-cadherin ([Clone 36]; BD Biosciences). Additional antibodies and small GTPase assays appearing in FIG. 7, as well as detailed protocols, can be found in Experimental Procedures.

Lentiviral Expression Constructs.

Tet-inducible lentivirus constructs modified from Invitrogen's Gateway lentiviral expression system (pLenti-CMV/TO-Neo-DEST#2, pLenti-CMV/TO-Puro-DEST#2, pLenti-RNAi-Puro-DEST#2, and pENTR-pTER') were kindly provided by Eric Campeau (Lawrence Berkeley National Laboratory, Berkeley, Calif.), while pENTR1A™ was available commercially (Invitrogen™). For forced expression of 14-3-3sigma (wild-type and ΔC mutant), pGEX-2TK-14-3-3sigma was used as a PCR template and was kindly provided by Michael Yaffe (Massachusetts Institute of Technology, Cambridge, Mass.; Addgene plasmid 11944), while for RNA interference, sense and antisense 61-mer oligonucleotides encoding previously validated shRNAs targeting 14-3-3sigma [42] or a scrambled sequence were used. The complete cloning strategy is provided in the Experimental Procedures. For lentiviral production, pLenti plasmids were cotransfected with pLP1, pLP2, and pLP-VSVG helper plasmids into 293FT host cells using Fugene6 transfection reagent (Roche).

Migration, Invasion, and Stellate Morphogenesis Assays.

Cell motility and invasion were measured as previously described using wound healing [11] and Boyden chamber invasion assays [8], respectively. For stellate morphogenesis assays, individual wells of a 24-well plate were coated with 2004, of lrECM (Cultrex™) and 40,000 cells were seeded on top of the polymerized gel. MCF10DCIS.com cells were cultured in MCF10A 3D assay media [91] for 4 days, then the number of projections per colony were manually counted in 5 independent fields per well. Results presented are the average number of projections per colony for 3 independent wells

Immunofluorescence.

Direct and indirect immunofluorescent labeling of formalin-fixed and 0.5% Triton X-100 permeabilized cells was performed according to standard protocols using antibody targeting 14-3-3sigma (C-18; Santa Cruz) and/or fluorescently conjugated phalloidin (Invitrogen). Nuclei were counterstained with DAPI (Invitrogen) and slides were mounted with Vectrashield (Vector Labs). For live-cell imaging, cells were seeded onto 1.0 chamber coverslips (VWR) previously coated overnight with bovine tail collagen (PureCol™), and only cells with marginal expression of fluorophores were selected for analysis to minimize artifacts resulting from disrupted endogenous actin dynamics. Live and fixed cells were imaged using a spinning disk (Yokogawa) confocal microscope based on a Zeiss Axiovert 200 microscope fitted with a cell incubator, under 63× Plan Apochromat (1.4 numeric aperture) oil immersion lens.

Image Analysis.

For F-actin quantification, phalloidin stains were converted into a binary mask such that pixels within the cell area have a value of 256 and pixels outside have a value of 0. The intensity of the phalloidin stain normalized to the mask area was calculated for at least 5 independent fields of cells using ImageJ software. For 3D image projections, data from confocal stacks (50 z-sections) spanning the entire cell volume were used to build a 3D rendering in Imaris software (Bitplane). To generate semi-quantitative heat maps, the pixel intensity of the mCherry fluorescence was subtracted from that of the YFP channel using ImageJ software, generating a 32-bit differential map to which a 16-color LUT was applied. The maxima and minima of each frame in the time course were uniformly adjusted to compensate for differences in background intensity and fluorophore intensity per cell while avoiding saturation in either channel, giving a positive value where there is higher 14-3-3sigma intensity, a negative value where there is higher F-actin intensity, and a neutral value where the intensities are equal.

Protein Purification and Bioconjugation.

Actin from young rabbit muscle tissue (Pel-Freez) was purified according to previously methods [93]. AlexaFluor488 conjugated actin was generated by labeling 1 mg of F-actin with AlexaFluor488 TFP ester (Invitrogen) according to the manufacturer's protocol, followed by subsequent cycles of depolymerization, polymerization, and depolymerization to ensure retention of activity following bioconjugation. For purification of recombinant proteins, pGEX-2TK 14-3-3sigma [36] (kindly provided by Michael Yaffe, Massachusetts Institute of Technology, Cambridge, Mass.; Addgene plasmid 11944) and the vector control were expressed in BL21(DE3)pLysS cells (Promega), and while cells were in log-growth phase, protein production was induced with 0.25 mM IPTG for 4 hours. Pelleted cells were lysed in ice-cold PBS containing 1% Triton X-100 and recombinant proteins were purified using columns packed with glutathione sepharose 4B according to the manufacturer's protocol (GE Healthcare).

In-Vitro Assays.

For GST pull-down assays, purified actin was diluted to a concentration of 0.1 mg/mL (2.38 μM) in GST-Fish buffer [11] and incubated with either GST or GST-14-3-3sigma (1:1 molar ratio), then recombinant proteins were pulled out of solution using glutathione sepharose 4B according to the manufacturer's suggestion (GE Healthcare), resolved by SDS-PAGE, and visualized using Coomassie stain (Invitrogen). For polymerization assays, 0.1 mg/mL actin was depolymerized into G-actin [93], incubated for 1 hour with recombinant proteins (1:1 molar ratio), then polymerized by addition of KCl to 50 mM, Mg²⁺ to 2 mM, and ATP to 1 mM concentrations. F-actin was separated from residual G-actin by fractionation at 150,000 g for 1.5 hours [93], and the F- and G-actin pools were resolved by SDS-PAGE as above. For fluorescent actin polymerization assays, 1 μM of 488-G-actin was pretreated with 4 μM of recombinant protein, then either polymerized in vitro and imaged by confocal microscopy as above, or introduced into cells permeabilized with 0.2 mg/mL saponin to measure in situ actin polymerization in the presence of ATP [43]. Following 488-G-actin incorporation, cells were fixed and the endogenous F-actin cytoskeleton was counterstained with phalloidin. Background intensity was adjusted by applying a threshold uniformly across all cell fields, and actin incorporation, quantified as the ratio of the 488-actin intensity divided by the phalloidin intensity, was calculated for at least 20 independent cell fields using ImageJ software.

Immunohistopathology.

14-3-3sigma immunohistochemistry was performed using a goat polyclonal antibody specific for the N-terminus (N-14; Santa Cruz Biotechnology) as previously described [9], either on T4-2 xenografts prepared by subcutaneous injection in the presence of lrECM [7], or on a tissue microarray prepared from 245 patients with invasive breast carcinoma which were treated with standard anthracycline-based adjuvant chemotherapy [49, 94]. Breast cancer tissue cores were analysed by three observers including two authors (FG & JSR—F), with observers blinded to the results of other immunohistochemical markers and patients' outcome [9]. For heat map projection, RGB images of T4-2 xenografts were converted to a 16-bit image, inverted, and a 16-color LUT was applied using ImageJ software.

cDNA Microarray Survival Analysis.

cDNA microarray data for patients comprising the Netherlands Cancer Institute (NKI) cohort was downloaded from Rosetta Inpharmatics server, and are described elsewhere [56]; clinical data for NKI patients is published elsewhere [58]. Clinical data and cDNA microarray data for patients comprising the UCSF cohort were downloaded from the LBNL server (<http://cancer.lbl.gov/breastcancer/>) and are described in detail elsewhere [57]. Patients were sorted based on 14-3-3sigma expression into 14-3-3sigma high (above median) and 14-3-3sigma low (below median) groups for Kaplan-Meier survival analysis.

Statistics.

All graphs, including Kaplan-Meier survival curves, were constructed using GraphPad Prism software (version 5.01). All statistics reported are the two-tailed, 95% confidence interval p-values.

### Example 7

### Targeting 14-3-3sigma Expression In Vivo

In a previous example, cells were shown to have decreased migration and invasion in culture assays when deficient in 14-3-3sigma expression. To test the validity of these findings in vivo, we generated xenografts in mice using T4-2 cells having disrupted expression of 14-3-3sigma (T4-2 sh-14-3-3σ) and compared the xenografts to those formed upon injecting control cells (T4-2 sh-scrambled). For xenografts, 5,000,000 cells in 50% Matrigel were injected subcutaneously into the rear flanks of 8 week old female BALB/c (nu/nu) mice. Tumor volumes were measured three times a week with a caliper until mice were sacrificed after 5-6 weeks of tumor growth. Tumors were fixed in 4% paraformaldehyde, paraffin embedded, sectioned, and stained with H&E (UCSF Mouse Pathology Core) for histological analysis. These experiments were performed using a protocol approved by the Lawrence Berkeley National Laboratory Animal Research and Welfare Committee.

H&E stains of tumors derived from T4-2 sh-scr and sh-14-3-3σ subcutaneous injection were examined to observe the morphology of the tumors and surrounding mouse stroma in independent tumor sections. It was found that tumors which formed from T4-2 sh-14-3-3σ xenografts showed a morphology characterized by more distinct margins and less perturbation to the adjacent normal tissue (resembling “carcinoma in situ), in comparison to control tumors which are very invasive (data not shown). These in vivo data indicate that disrupting 14-3-3sigma expression can inhibit tumor progression from in situ to invasive carcinoma, an important stage in the transition towards advanced, metastatic disease.

### Example 8

### Detecting 14-3-3sigma for Diagnosis and Prognosis

We demonstrated that 14-3-3sigma, when expressed at high levels in breast tumors, effectively identifies basal-like breast cancer and correlates with poor clinical outcome. As the vast majority of patients surveyed which have 14-3-3sigma expression and lack estrogen receptor are of basal-like molecular subtype, one can predict that a patient who has a biopsy taken which is found to be ER-negative and 14-3-3sigma positive breast cancer will most likely have basal-like breast cancer.

As basal-like breast cancers typically are ER/PR/HER2 “triple negative”, these tumors are not predicted to respond to current therapies targeting these molecules. Additionally, basal-like breast tumors have poor 5-year clinical outcome. As such, 14-3-3sigma immunohistochemical staining can be employed as a diagnostic and predictive tool, whereby a patient biopsy can be stained and if high expression of 14-3-3sigma and absence of ER is confirmed, it can be concluded by the clinician that the patient would most likely not respond to tamoxifen, HER2, or related therapies and will most likely have poor clinical outcome. Recommendations can be made for the patient to be prioritized for more aggressive treatment with other therapies (if possible). In contrast, if 14-3-3sigma is absent and ER is present in the biopsy, the patient would more likely have better response to tamoxifen or related therapies and most likely better clinical outcome.

In such an example, immunohistochemical staining of a non-malignant breast tissue can be employed as a standard, whereby 14-3-3sigma staining intensity in tumors that is greater than or equal to the intensity observed in myoepithelial cells of the normal breast can be scored as positive.

### Example 9

### Targeting 14-3-3sigma-Mediated Actin Inhibition as a Novel Anti-Metastasis Therapeutic

Assuming the compounds (or their derivatives) described herein can freely enter cells/tissues, one would first test whether when the compound is added to cells in culture, there is increased actin polymerization within the cell. This can be done in a rather high-throughput manner by fixing the cells, staining with a small molecule (e.g., phalloidin) which binds to (polymerized) F-actin, then imaging the cells by confocal microscopy. All candidates could be introduced at once to cells grown in a 96-well plate, and F-actin staining could be quantified using a high content immunofluorescence machine, such as Cellomics.

To verify activity of drugs which inhibit 14-3-3sigma::actin interaction (either directly or indirectly), purified 14-3-3sigma can be pretreated with the drug, added to purified G-actin, and then the G-actin polymerized by adding ATP, KCl, and MgCl₂. If the drug inhibits 14-3-3 sigma, one would expect to see robust polymerization of actin in the presence of the drug, while in the absence of the drug, 14-3-3sigma would still be able to inhibit actin polymerization.

To verify drugs which stabilize 14-3-3sigma interaction with other proteins, purified 14-3-3sigma and a purified 14-3-3sigma ligand (such as Raf) could be incubated together, the drug added, then 14-3-3sigma and any interacting Raf pulled out of solution by immunoprecipitation. If the drug stabilizes 14-3-3sigma interaction with ligands, then more Raf would be pulled down with 14-3-3sigma in its presence relative to performing the same experiment without the drug included.

To verify drugs which prevent 14-3-3sigma dimerization, recombinant 14-3-3sigma can be treated with the drug, then resolved by native polyacrylamide gel electrophoresis. If the drug inhibits dimerization, a stronger band corresponding to the monomeric 14-3-3 molecular weight would be apparent, coinciding with a weaker band at the molecular weight of dimeric 14-3-3sigma, relative to untreated recombinant protein.

To verify drugs that act by modulating 14-3-3sigma gene expression (i.e. 14-3-3sigma promoter methylating agents or RNAi agents), the agent is introduced to cells, and the levels of 14-3-3sigma RNA and protein relative to an untreated control are quantified by q-PCR and western blot analysis, respectively. To verify the activity of drugs that promote 14-3-3sigma proteasomal degradation, the drug is introduced and the levels of 14-3-3 protein are measured by western blot analysis relative to an untreated control. These agents should not influence RNA levels (measured by q-PCR).

Once the actin inhibition and mechanism of action is known, the drugs can be tested for efficacy as chemotherapeutics. To test whether the drug inhibits cell migration, cells are grown to confluent monolayers, scratched with a pipet tip, and the rate by which cells migrate into the gap is quantified in the presence and absence of the drug. To test whether the drug inhibits invasion, cells are seeded on top of Matrigel™ coated transwell inserts containing 8 μm pores. The rate by which cells degrade through the Matrigel™ and migrate through the pores is quantified over time, in the presence and absence of the drug.

If the drug inhibits cell migration and invasion (important prerequisites to metastasis), one would next test whether the drugs inhibit lung metastasis of a highly metastatic cell line (such as MDA-MB-231) when injected into the tail vein of immunocompromised mice. One would test a wide variety of methods of drug delivery, including pretreating cells with the agent or co-injection of the drug with cells into the tail vein, followed by scheduled injections for a period of several weeks according to standard protocols. At the end point of the experiment, the mice would be sacrificed, then the lung sections stained using an antibody specific for human cytokeratins (to quantify the rate of lung metastasis of the human MDA-MD-231 cells). If the drug inhibits metastasis in circulating cells, there should be a dramatic decrease in the rate of lung metastasis in mice administered with the drug. Other metastasis sites, such as the brain and bone, can similarly be quantified.

To test whether the drug inhibits primary tumor dissemination/metastasis, xenografts could be performed by injecting breast cancer cells into mice to form a primary tumor, and the rate of metastasis could be monitored at time intervals following the initial appearance of a tumor beyond a given size. Many methods could be used. For example, one would inject cells into the “cleared” mammary gland fat pad of immunocompromised/athymic mice. Once the tumor establishes the drug can be administered (as optimized in tail vein injection studies), and the frequency of metastasis to distant organs quantified as for tail vein injection studies. This can be done either following removal of the tumor (adjuvant treatment) or with the tumor present (neoadjuvant treatment), and the frequency of distant recurrence, tumor shrinkage, and overall survival (in the presence or absence of drug) would be experimental endpoints. Other in vivo metastasis assays could be performed as well.

If the drug is found to inhibit metastasis, toxicity in mice (similar to a human Phase I trial) and other preclinical validation would be performed, following the standard drug development pipeline.

The examples and methods described herein are meant to exemplify and illustrate the invention and should in no way be seen as limiting the scope of the invention. Any patents or publications mentioned in this specification are indicative of levels of those skilled in the art to which the patent pertains and are hereby incorporated by reference to the same extent as if each was specifically and individually incorporated by reference.

TABLE 1

| | Parameter | | N | 14-3-3σ negative | 14-3-3σ strong | P value |
|---|---|---|---|---|---|---|
| Clinical parameters | Size - TNM | | 198 | | | NS § |
| | | T1 | | 84 | 17 | |
| | | T2 | | 70 | 13 | |
| | | T3 | | 12 | 2 | |
| | Grade | | 194 | | | 0.0010 § |
| | | 1 | | 18 | 0 | |
| | | 2 | | 53 | 3 | |
| | | 3 | | 91 | 29 | |
| | Type | | 197 | | | NS § |
| | | IDC | | 118 | 26 | |
| | | ILC | | 29 | 1 | |
| | | Mixed | | 12 | 3 | |
| | | Other | | 6 | 2 | |
| | LV1 | | 197 | | | NS ‡ |
| | | − | | 53 | 11 | |
| | | + | | 112 | 21 | |
| | LN mets | | 192 | | | NS ‡ |
| | | − | | 53 | 12 | |
| | | + | | 107 | 20 | |
| | ER | | 195 | | | <0.0001 ‡ |
| | | − | | 19 | 17 | |
| | | + | | 144 | 15 | |
| | PR | | 195 | | | <0.0001 ‡ |
| | | − | | 33 | 19 | |
| | | + | | 130 | 13 | |
| | HER2 | | 193 | | | NS ‡ |
| | | − | | 135 | 30 | |
| | | + | | 26 | 2 | |
| | Ki-67 | | 186 | | | 0.0001 § |
| | | <10% | | 69 | 8 | |
| | | 10-30% | | 71 | 11 | |
| | | >30% | | 15 | 12 | |
| Basal and | EGFR | | 198 | | | <0.0001 ‡ |
| luminal markers | | − | | 160 | 21 | |
| | | + | | 6 | 11 | |
| | Ck 14 | | 197 | | | <0.0001 ‡ |
| | | − | | 160 | 22 | |
| | | + | | 5 | 10 | |
| | Ck 5/6 | | 191 | | | <0.0001 ‡ |
| | | − | | 155 | 17 | |
| | | + | | 7 | 12 | |
| | Ck 17 | | 195 | | | <0.0001 ‡ |
| | | − | | 153 | 20 | |
| | | + | | 10 | 12 | |
| | Caveolin 1 | | 198 | | | <0.0001 ‡ |
| | | − | | 158 | 22 | |
| | | + | | 8 | 10 | |
| | Caveolin 2 | | 179 | | | 0.0023 ‡ |
| | | − | | 146 | 22 | |
| | | + | | 5 | 6 | |
| | FOXA1 | | 158 | | | 0.0318 ‡ |
| | | − | | 28 | 10 | |
| | | + | | 107 | 13 | |
| Subtypes | Nielsen groups | | 187 | | | <0.0001 § |
| | | Basal | | 7 | 16 | |
| | | Luminal | | 122 | 14 | |
| | | HER2 | | 27 | 1 | |
| | Triple negative | | 195 | | | <0.0001 ‡ |
| | | No | | 152 | 19 | |
| | | Yes | | 11 | 13 | |

Ck: cytokeratin;
ER: estrogen receptor;
IDC: invasive ductal carcinoma;
ILC: invasive lobular carcinoma;
LN mets: lymph node metastasis;
LVI: lympho-vascular invasion;
Nielsen groups: immunophenotypic groups defined based upon the expression of ER, HER2, Ck 5/6 and EGFR (Nielsen et al., 2004);
PR: progesterone receptor
§: χ² p values (two-tailed, 95% confidence interval)
‡: Fisher's exact test p values (two-tailed, 95% confidence interval)

1. An assay for diagnosis of basal-like breast cancer comprising the steps of (a) obtaining a breast tissue sample, and (b) detecting a positive and/or elevated level of 14-3-3sigma expression in said breast tissue sample as compared to a reference, whereby such positive and/or elevated detection is a diagnosis of basal-like breast cancer.

2. The assay of claim 1 wherein the detecting is by fluorescence in situ hybridization (FISH) to detect 14-3-3sigma expression levels.

3. The assay of claim 1 wherein the detecting is by polymerase chain reaction (PCR) assay to detect 14-3-3sigma expression levels.

4. The assay of claim 1 wherein the detecting is by RT-PCR to detect 14-3-3sigma transcription levels.

5. The assay of claim 1 wherein the detecting is by an immunohistochemical analysis to detect 14-3-3sigma protein levels.

6. The assay of claim 5 wherein a positive level of 14-3-3sigma is detected if the 14-3-3sigma level detected in said breast tissue sample is greater than or equal to the levels observed in a myoepithelial cell tissue sample of the normal breast.

7. A kit containing reagents to carry out the assay of claim 1.

8. A method for identifying an ER-negative cancer patient with poor prognosis, comprising: (a) measuring the expression level of 14-3-3sigma (SFN) gene in a sample from the patient; and (b) comparing the expression level of said gene from the patient with the expression level of the gene in a normal tissue sample or a reference expression level, whereby an increased expression level in said gene indicates a patient with poor prognosis.

9. A compound to treat patients with elevated 14-3-3sigma expression, wherein the compound is a 14-3-3sigma inhibitor.

10. The compound of claim 9 wherein the inhibitor is a small molecule that interferes with 14-3-3sigma function.

11. The compound of claim 9 wherein the inhibitor is a viral vector producing a nucleic acid sequence that inhibits 14-3-3sigma.

12. The compound of claim 9 wherein the inhibitor is an aptamer.

13. The compound of claim 9 wherein the inhibitor is an antisense oligonucleotide.

14. The compound of claim 9 wherein the inhibitor is a siRNA oligonucleotide.

15. The compound of claim 14 wherein the siRNA oligonucleotide has a sequence of SEQ ID NOS: 7 and 8.

16. (canceled)

17. (canceled)

18. (canceled)

19. (canceled)

20. (canceled)

21. A method for diagnosis of basal-like cancer comprising the steps of (a) obtaining a tissue sample, and (b) detecting a positive and/or elevated level of 14-3-3sigma expression in said tissue sample as compared to a reference, whereby such positive and/or elevated detection is a diagnosis of basal-like cancer.

22. The method of claim 21, wherein said cancer is breast, ovary, prostate, lung, pancreatic, bladder, colorectal, endometrial, head and neck, skin, squamous, or other epithelial or myoepithelial cancer.

23. The method of claim 22, wherein said cancer is breast, pancreatic, colorectal, head and neck or squamous cancer.
